(19) United States
(12) Patent Application Publication
(10) Pub. No.: US 2021/0369995 A1
(43) Pub. Date: Dec. 2, 2021

(54) METHODS AND SYSTEMS FOR A MEDICAL GAS QUALITY MONITOR

(71) Applicant: GE Precision Healthcare LLC, Milwaukee, WI (US)

(72) Inventor: Russell Kuzelka, McFarland, WI (US)

(21) Appl. No.: 16/884,831

(22) Filed: May 27, 2020

(51) Int. Cl. A61M 16/00; G01N 33/497; G01N 33/00; G01N 15/06

(52) U.S. Cl. CPC A61M 16/0003; G01N 33/497; G01N 33/004; G01N 2015/0088; G01N 15/06; A61M 16/0051; G01N 33/0047

(57) ABSTRACT

Various methods and systems are provided for determining a quality of a medical gas flow. In one example, a method for a medical gas quality monitoring system includes obtaining measurements of a medical gas via a plurality of sensors, the plurality of sensors including at least one of a humidity sensor, a particulate matter sensor, a carbon dioxide sensor, and a total volatile organic compound (tVOC) sensor, determining a gas quality index of the medical gas based on the obtained measurements, and outputting the determined gas quality index.

### FIELD

Embodiments of the subject matter disclosed herein relate to gas delivery systems, and more particularly, to devices for monitoring medical gas supplied from the gas delivery systems.

### BACKGROUND

Healthcare facilities, such as hospitals, include medical gas pipelines and gas-holding cylinders that deliver different types of medical gases (e.g., oxygen, nitrogen, carbon dioxide, and nitrous oxide) to various locations throughout the facility. For example, the medical gas pipelines may supply the medical gases from source equipment (e.g., gas tanks, pumps, compressors, dryers, receivers, and manifolds) at a centralized location to gas delivery systems at a patient care location via a network of pipes and service outlets, whereas gas-holding cylinders may store the medical gases at the patient care location. The gas delivery system may in turn provide the medical gases to a patient, such as to provide anesthesia (e.g., when the gas delivery system is configured as an anesthesia machine) and/or to assist in respiration (e.g., when the gas delivery system is configured as a ventilator).

### BRIEF DESCRIPTION

In one embodiment, a method for a medical gas quality monitoring system includes obtaining measurements of a medical gas via a plurality of sensors, the plurality of sensors including at least one of a humidity sensor, a particulate matter sensor, a carbon dioxide sensor, and a total volatile organic compound (tVOC) sensor, determining a gas quality index of the medical gas based on the obtained measurements, and outputting the determined gas quality index. In this way, the medical gas quality monitoring system may output an indication of a quality of a medical gas provided to a patient, and the output indication may be reviewed by a care provider, for example.

It should be understood that the brief description above is provided to introduce in simplified form a selection of concepts that are further described in the detailed description. It is not meant to identify key or essential features of the claimed subject matter, the scope of which is defined uniquely by the claims that follow the detailed description. Furthermore, the claimed subject matter is not limited to implementations that solve any disadvantages noted above or in any part of this disclosure.

### BRIEF DESCRIPTION OF THE DRAWINGS

The present disclosure will be better understood from reading the following description of non-limiting embodiments, with reference to the attached drawings, wherein below:

FIG. 1 schematically shows an embodiment of an anesthesia machine.

FIG. 2 schematically shows an embodiment of a gas quality monitor that may be included in a medical gas quality monitoring system.

FIG. 3 schematically shows a first embodiment of a medical gas quality monitoring system that may be installed in the anesthesia machine of FIG. 1.

FIG. 4 schematically shows a second embodiment of a medical gas quality monitoring system that may be installed in the anesthesia machine of FIG. 1.

FIG. 5 schematically shows a third embodiment of a medical gas quality monitoring system that may be installed in the anesthesia machine of FIG. 1.

FIG. 6 schematically shows a fourth embodiment of a medical gas quality monitoring system that may be installed in the anesthesia machine of FIG. 1.

FIG. 7 schematically shows a fifth embodiment of a medical gas quality monitoring system that may be installed in the anesthesia machine of FIG. 1.

FIGS. 8A and 8B show a flow chart of an example method for monitoring a quality of a medical gas and detecting potential contaminants.

FIG. 9 shows an example chart of potential particulate contaminants according to size.

FIG. 10 is a first prophetic example timeline for detecting medical gas contamination based on outputs from sensors of a medical gas quality monitoring system.

FIG. 11 is a second prophetic example timeline for detecting medical gas contamination based on outputs from sensors of a medical gas quality monitoring system.

FIG. 12 is a third prophetic example timeline for detecting medical gas contamination based on outputs from sensors of a medical gas quality monitoring system.

### DETAILED DESCRIPTION

The following description relates to various embodiments for monitoring a flow of medical gas provided to a patient via a medical gas flow device, such as an anesthesia machine or ventilator. During operation, the medical gas flow device typically receives the medical gas (e.g., oxygen, nitrogen, nitrous oxide, air, carbon dioxide, or a combination thereof) from a centralized location that is remote from a patient care location, and thus, remote from the medical gas flow device. For example, the medical gas may be carried from the centralized location to the patient care location via a medical gas pipeline. In some examples, the medical gas delivered by the medical gas pipeline may become contaminated, such as by water vapor, oil, particulates, or microbes, for example, thereby reducing its quality. The centralized location may include gas quality monitoring devices that monitor for gas contaminants and gas composition and alert personnel at the centralized location to any deviations in gas quality, at least in some examples. However, these devices do not alert care providers to these degradations in gas quality and do not detect contaminants introduced in the medical gas upstream of the centralized location, including at the medical gas flow device itself. Thus, an operator of the medical gas flow device may continue to flow the contaminated medical gas through the medical gas flow device. The contaminated gas may degrade components of the gas delivery system, for example, leading to shutdown of the device, high maintenance costs, and increased operator frustration. As another example, it is desirable to reduce patient exposure to lower quality medical gas.

Thus, according to embodiments disclosed herein, a medical gas quality monitoring system is provided to determine a gas quality of a medical gas at a patient care location. For example, the medical gas provided by the medical gas pipeline may first flow through one or more gas quality monitors before flowing to a patient. The one or more gas quality monitors may include a plurality of sensors in order to detect water vapor (e.g., via a humidity sensor), chemical or hydrocarbon contamination (e.g., via a volatile organic compound sensor), particulate contamination (e.g., via a particulate matter sensor), and/or gas composition (e.g., via a carbon dioxide sensor). A controller of the medical gas quality monitoring system may monitor signals received from the plurality of sensors to determine a gas quality index, which may be output to a display. Further, the controller may use the signals received from the plurality of sensors in various combinations to determine if contaminants are present and to distinguish a type of contamination present (e.g., water vapor, biological, non-biological particulate, or chemical). In some examples, the controller may perform a disinfection routine (e.g., responsive to biological contamination being present) or a flushing routine (e.g., responsive to chemical contamination being present) to at least reduce an extent of the contamination.

The embodiments disclosed herein may provide several advantages. For example, the embodiments disclosed herein may provide for real-time monitoring of a quality of a medical gas provided to a medical gas flow device at a patient care location, thereby reducing equipment and patient exposure to a contaminated or otherwise degraded medical gas. As another example, the controller of the medical gas quality monitoring system may communicate the medical gas quality and any detected degradation to the operator of the medical gas flow device and a log so that the medical gas quality can be tracked. For example, tracking the medical gas quality may enable cross-correlation between gas delivery system maintenance and/or particular gas vendors, for example.

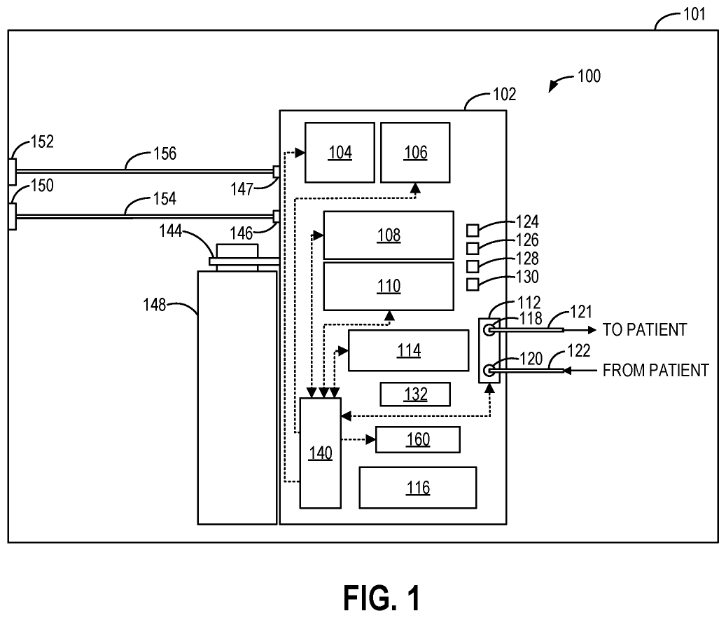

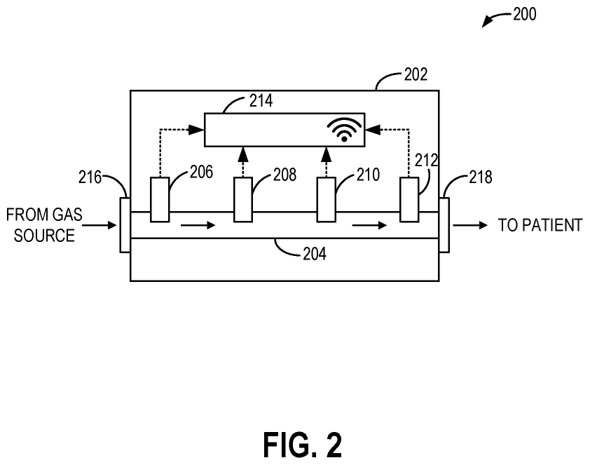

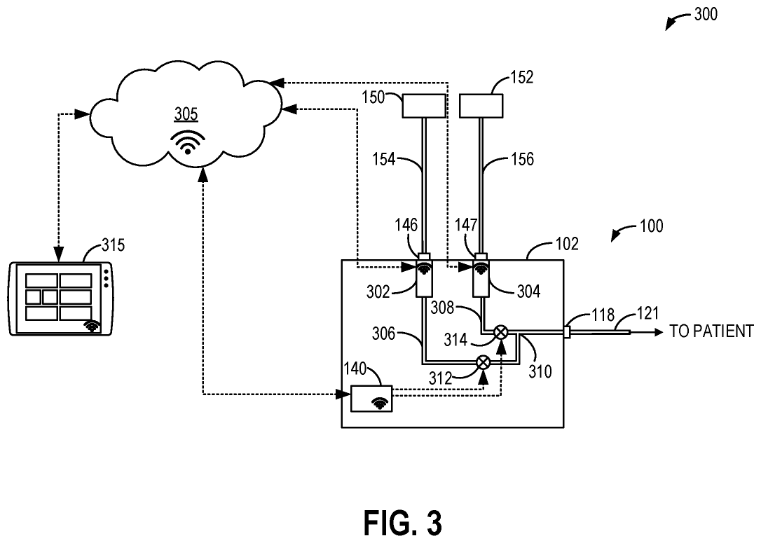

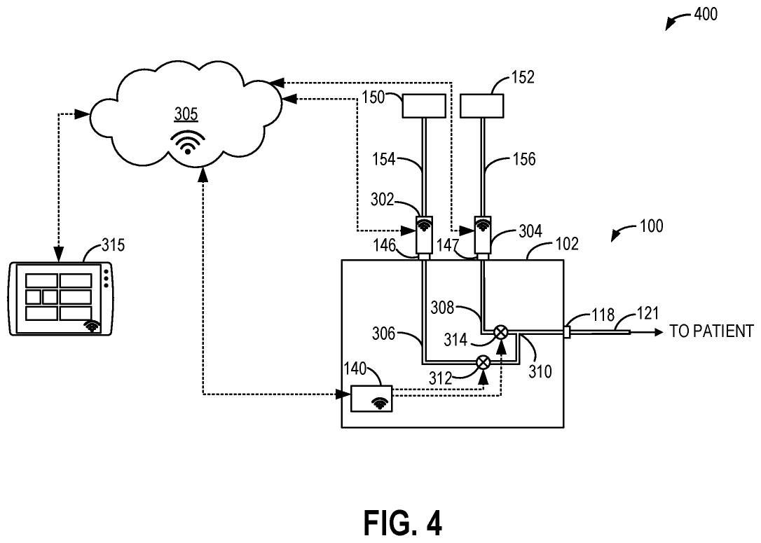

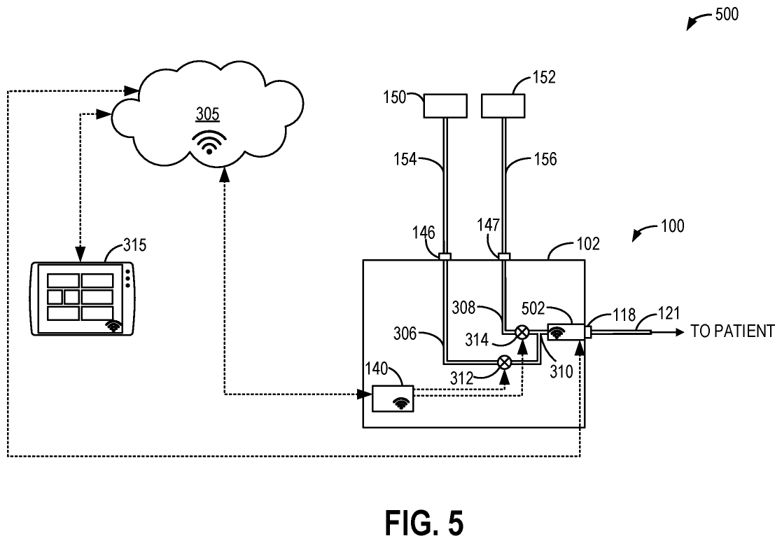

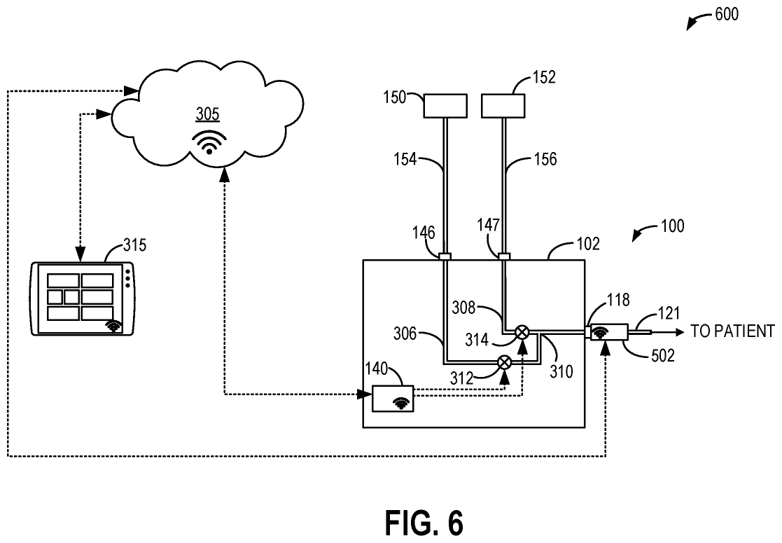

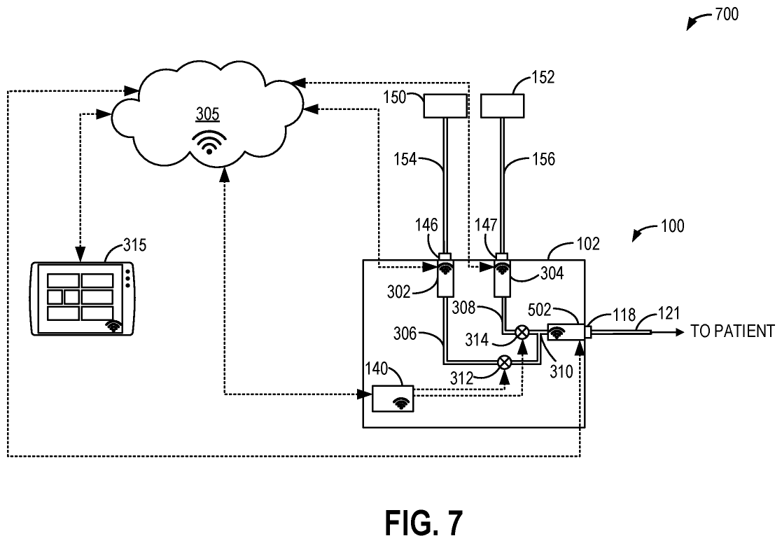

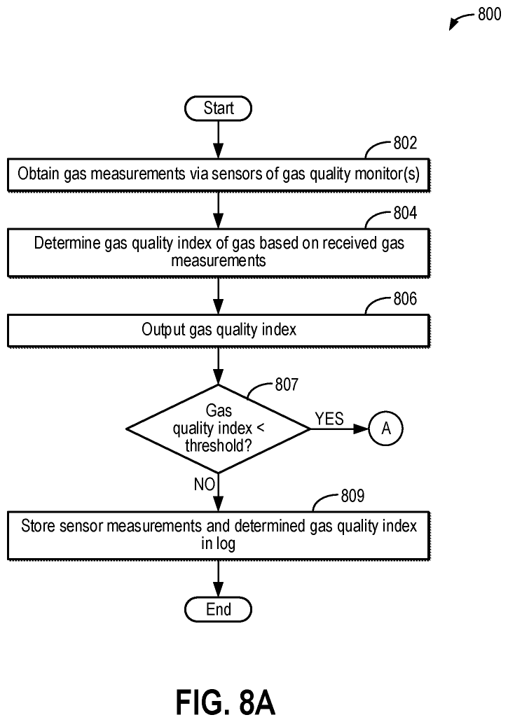

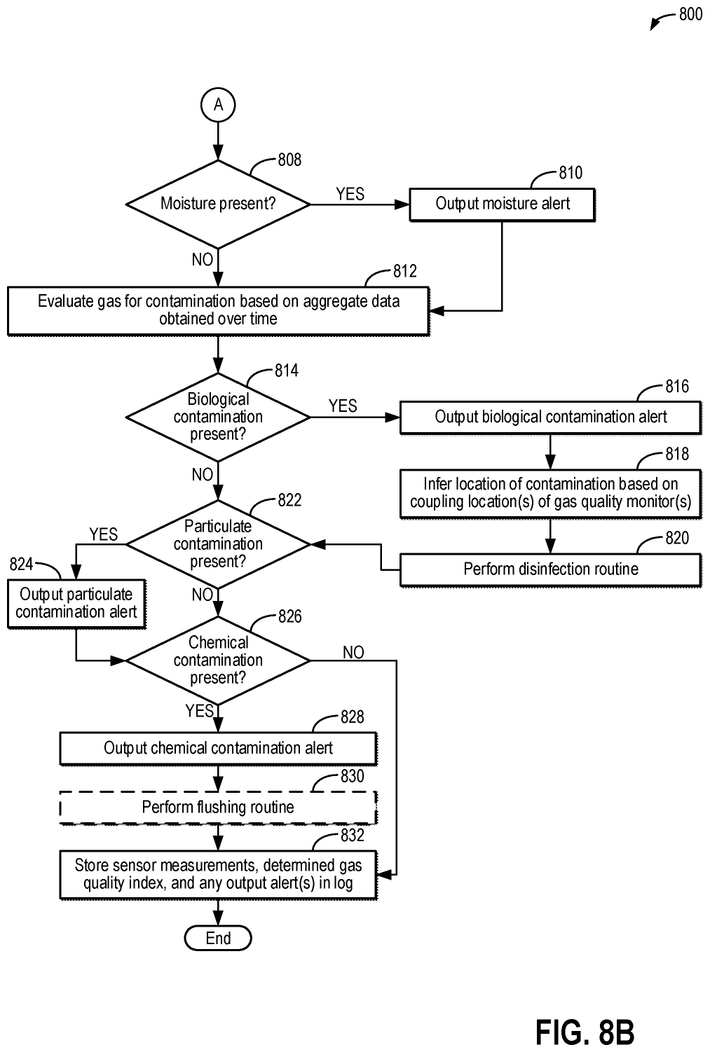

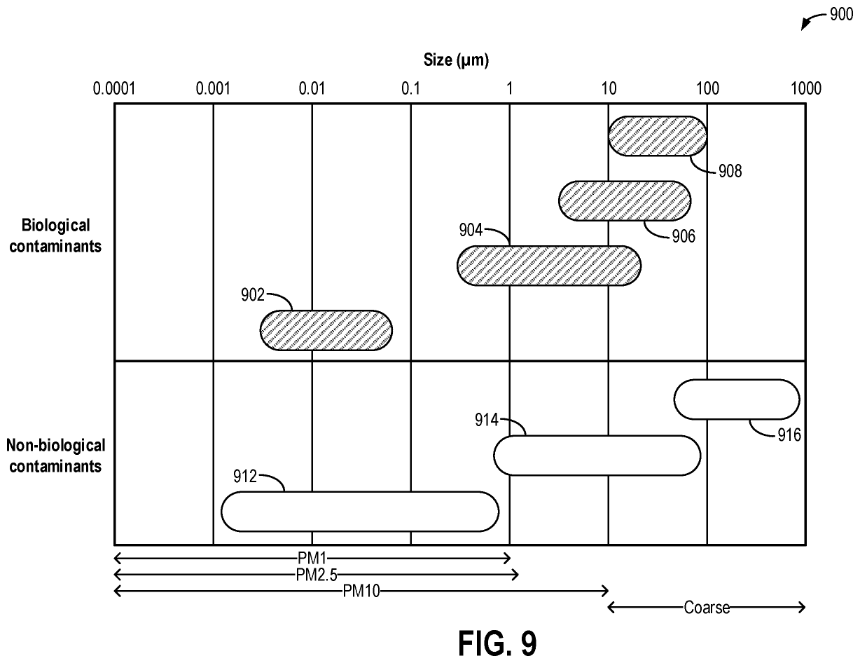

FIG. 1 shows an anesthesia machine as an example of a medical gas flow device (e.g., gas delivery system or device), according to an embodiment of the disclosure. FIG. 2 shows an embodiment of a gas quality monitor that may be included in a medical gas quality monitoring system that is used to monitor a gas quality index of a gas flow provided to or from a medical gas flow device, such as the anesthesia machine of FIG. 1. In particular, the gas quality monitor may be configured to measure quantities within a gas flow that enable the determination of the gas quality index of the gas flow. FIG. 3 shows a first embodiment of the medical gas quality monitoring system installed in the anesthesia machine of FIG. 1, the first embodiment including two inlet gas quality monitors that are integrated within the anesthesia machine (e.g., one for each gas inlet of the anesthesia machine). FIG. 4 shows a second embodiment of the medical gas quality monitoring system, the second embodiment including two inlet gas quality monitors that are external to the anesthesia machine. FIG. 5 shows a third embodiment of the medical gas quality monitoring system, the third embodiment including one outlet gas quality monitor that is internal to the anesthesia machine. FIG. 6 shows a fourth embodiment of the medical gas quality monitoring system, the fourth embodiment including one outlet gas quality monitor that is external to the anesthesia machine. FIG. 7 shows a fifth embodiment of the medical gas quality monitoring system, the fifth embodiment including two inlet gas quality monitors and one outlet gas quality monitor. A controller may utilize sensor output received from each gas quality monitor included in the gas quality monitoring system to determine the gas quality index of the gas provided to and/or from the anesthesia machine and determine if contamination is present according to the exemplary method shown in FIGS. 8A and 8B. FIG. 9 shows an example chart of potential particulate contaminants according to size, including biological contaminants and non-biological particulate contaminants. Prophetic examples of combining sensor outputs from the gas quality monitor to detect and distinguish a type of contamination are shown in FIGS. 10-12.

Turning now to the figures, FIG. 1 schematically shows an example anesthesia machine 100. The anesthesia machine 100 is one embodiment of a medical gas flow device that may be used to supply medical gas to a patient. The anesthesia machine 100 is positioned within a patient care location 101, which may be a hospital ward, operating theater, patient room, or other location within a healthcare facility, for example. The anesthesia machine 100 includes a housing (or frame) 102. In some embodiments, the housing 102 may be supported by casters, where the movement of the casters may be controlled (e.g., stopped) by one or more locks. In some examples, the housing 102 may be formed of a plastic material (e.g., polypropylene). In other examples, the housing 102 may be formed of a different type of material (e.g., metal, such as steel).

The anesthesia machine 100 also includes an anesthesia display device 104, a patient monitoring display device 106, a respiratory gas module 108, one or more patient monitoring modules, such as a patient monitoring module 110, a ventilator 112 (explained in more detail below), an anesthetic vaporizer 114, and an anesthetic agent storage bay 116. The anesthesia machine 100 may further include a main power indicator 124, a system activation switch 126 (which, in one example, permits gas flow when activated), an oxygen flush button 128, and an oxygen control 130. The anesthetic vaporizer 114 may vaporize the anesthetic agent and combine the vaporized anesthetic agent with one or more medical gases (e.g., oxygen, air, nitrous oxide, or combinations thereof), which may then be delivered to a patient.

The anesthesia machine 100 may additionally include an integrated suction, an auxiliary oxygen flow control, and various other components for providing and/or controlling a flow of the one or more medical gases to the patient. In the embodiment shown, the anesthesia machine 100 includes a first pipeline connector 146 and a second pipeline connector 147 to facilitate coupling of the anesthesia machine to pipeline gas sources. Specifically, the first pipeline connector 146 is coupled to a first pipeline gas supply outlet 150 via tubing 154, and the second pipeline connector 147 is coupled to a second pipeline gas supply outlet 152 via tubing 156. For example, the pipeline gas supply outlets 150 and 152 may be included in a wall mount, a ceiling mount, a ceiling column, a bedhead unit, or another mounting locations. Each pipeline gas supply outlet 150 and 152 may provide medical gas originating from a pipeline gas supply at a central medical gas distribution system that is remote from the patient care location 101, as will be further described below. Further, although two pipeline connectors 146 and 147 and two pipeline gas supply outlets 150 and 152 are shown in FIG. 1, in other embodiments, more or fewer pipeline gas connectors and/or pipeline gas supply outlets may be included.

Each of the pipeline gas supply outlets may deliver a different type of medical gas, which may be coupled to a dedicated pipeline gas connector for that particular type of medical gas (e.g., oxygen, air, nitrous oxide, nitrogen, or carbon dioxide). As one illustrative example, the first pipeline gas supply outlet 150 delivers oxygen, which is received at the first pipeline connector 146 via the tubing 154, and the second pipeline gas supply outlet 152 delivers medical air, which is received at the second pipeline connector 147 via the tubing 156. Additionally, the anesthesia machine 100 includes a cylinder yoke 144, via which one or more gas-holding cylinders 148 may be coupled to the anesthesia machine. Thus, through pipeline connections and/or cylinder connections, gas may be provided to the anesthesia machine, where the gas may include (but is not limited to) medical air, oxygen, nitrogen, and nitrous oxide.

The gas that enters the anesthesia machine 100 may mix with the vaporized anesthetic agent at the anesthetic vaporizer 114, as described above, before being supplied to a patient via the ventilator 112. The anesthesia machine may also include a serial port, a collection bottle connection, and a cylinder wrench storage area. Further, in some embodiments, the anesthesia machine may include an anesthesia gas scavenging system 132 that may use an adsorbent (e.g., activated carbon) that adsorbs anesthetic agent exhaled from the patent.

The ventilator 112 may include an expiratory check valve at an expiratory port 120, an expiratory flow sensor at the expiratory port 120, an inspiratory check valve at an inspiratory port 118, an inspiratory flow sensor at the inspiratory port 118, an absorber canister, a manual bag port, a ventilator release, an adjustable pressure-limiting valve, a bag/vent switch, and a bellows assembly. When a patient breathing circuit is coupled to the ventilator 112, breathing gases (e.g., air, oxygen, and/or nitrous oxide mixed with vaporized anesthetic agent) exit the anesthesia machine from the inspiratory port 118 and travel to the patient via an inspiratory gas passage 121 coupled to the inspiratory port 118. Expiratory gases from the patient re-enter the anesthesia machine via an expiratory gas passage 122 coupled to the expiratory port 120, where carbon dioxide may be removed from the expiratory gases via the absorber canister.

During operation of the anesthetic vaporizer 114, an operator (e.g., an anesthesiologist) may adjust an amount of vaporized anesthetic agent that is supplied to the patient by adjusting a flow rate of gases from the gas source(s) (e.g., the pipeline gas supply) to the vaporizer. The flow rate of the gases from the gas source to the vaporizer may be adjusted by the operator via adjustment of one or more flow adjustment devices. For example, the flow adjustment devices may include analog and/or digital adjustment dials and/or other user input devices configured to actuate one or more flow control valves of anesthesia machine 100. In some embodiments, a first flow control valve may be positioned between the gas source(s) and the anesthetic vaporizer 114 and may be actuatable via the flow adjustment devices to a fully opened position, a fully closed position, and a plurality of positions between the fully opened position and the fully closed position.

Anesthesia machine 100 may additionally include one or more valves configured to bypass gases from the gas source(s) around the anesthetic vaporizer 114. The valves may enable a first portion of gases to flow directly from the gas source to the inspiratory port 118 and a second portion of gases to flow from the gas source through the anesthetic vaporizer 114 to mix with the vaporized anesthetic agents prior to flowing to the inspiratory port 118. By adjusting a ratio of the first portion of gases relative to the second portion of gases, the operator may control a concentration of vaporized anesthetic agent administered to the patient via the inspiratory port 118.

Further, the adjustments described above may be facilitated at least in part based on output from the respiratory gas module 108. The respiratory gas module 108 may be configured to measure various parameters of the gases exiting the vaporizer and/or being provided to the patient. For example, the respiratory gas module 108 may measure the concentrations of carbon dioxide, nitrous oxide, and the anesthetic agent provided to the patient. Further, the respiratory gas module 108 may measure respiration rate, minimum alveolar concentration, patient oxygen, and/or other parameters. The output from the respiratory gas module 108 may be displayed via a graphical user interface on a display device (e.g., the anesthesia display device 104 and/or the patient monitoring display device 106) and/or used by a controller to provide closed-loop feedback control of the amount of anesthesia provided to the patient.

The inspiratory gas passage 121 may be coupled between an airway of the patient (e.g., via a breathing mask positioned to enclose the mouth and/or nose of the patient or via a tracheal intubation tube) and the inspiratory port 118. Gases (e.g., the one or more medical gases, or a mixture of the one or more medical gases and vaporized anesthetic agent from the anesthetic vaporizer 114) may flow from the inspiratory port 118, through the inspiratory gas passage 121, and into the airway of the patient, where the gases are absorbed by the lungs of the patient. By adjusting the concentration of vaporized anesthetic agent in the gases as described above, the operator may adjust a degree to which the patient is anesthetized.

During conditions in which the inspiratory gas passage 121 is coupled to the airway, the anesthetic agent and/or fresh gas (without the anesthetic agent) may flow into the airway of the patient (e.g., through inhalation) via the inspiratory port 118 and the inspiratory check valve. As an example, the inspiratory check valve may open automatically (e.g., without input or adjustment by the operator) in response to inhalation by the patient and may close automatically in response to exhalation by the patient. Similarly, the expiratory check valve may open automatically in response to exhalation by the patient and may close automatically in response to inhalation by the patient.

In some embodiments, the operator may additionally or alternatively control one or more operating parameters of the anesthesia machine 100 via an electronic controller 140 of the anesthesia machine 100. The controller 140 includes a processor operatively connected to a memory. The memory may be a non-transitory computer-readable medium and may be configured to store computer executable code (e.g., instructions) to be processed by the processor in order to execute one or more routines, such as those described herein. The memory may also be configured to store data received by the processor. The controller 140 may be communicatively coupled (e.g., via wired or wireless connections) to one or more external or remote computing devices, such as a hospital computing system, and may be configured to send and receive various information, such as electronic medical record information, procedure information, and so forth. The controller 140 may also be electronically coupled to various other components of the anesthesia machine 100, such as the anesthetic vaporizer 114, the ventilator 112, the respiratory gas module 108, the anesthesia display device 104, and the patient monitoring display device 106.

Further, in the embodiment shown, the anesthesia machine 100 includes an ultraviolet germicidal irradiation (UVGI) system 160. The UVGI system 160 includes a plurality of UV light sources, which may be light-emitting diodes (LEDs) or mercury-vapor lamps, for example, that emit light in the ultraviolet (UV) wavelength range. In particular, the light emitted by the plurality of UV light sources of the UVGI system 160 may be short-wavelength UV-C light (e.g., having a wavelength between 100 and 280 nm). The plurality of UV light sources may be distributed throughout the anesthesia machine 100 and may be positioned to direct UV light toward gas flow passages, valves within the gas flow passages, and gas flow passage connectors. The UV light may kill or inactivate microorganisms, such as bacteria, viruses, and molds, on the irradiated surfaces. The controller 140 may activate the UVGI system 160, causing the plurality of UV light sources to emit UV light, according to a disinfection schedule. As another example, the controller 140 may activate the UVGI system 160 responsive to detected biological contamination, as will be further described below with respect to FIGS. 8A and 8B.

The controller 140 receives signals from the various sensors of the anesthesia machine 100 and employs the various actuators of the anesthesia machine 100 to adjust operation of the anesthesia machine 100 based on the received signals and instructions stored on the memory of the controller. For example, the flow of gases to the inspiratory port 118 may be controlled via an input device (e.g., keyboard, touchscreen, etc.) coupled to the electronic controller of the anesthesia machine 100. The controller 140 may display operating parameters of the anesthesia machine 100 via the anesthesia display device 104 and/or the patient monitoring display device 106. The controller may receive signals (e.g., electrical signals) via the input device and may adjust operating parameters of the anesthesia machine 100 in response (e.g., responsive) to the received signals.

As one example, the operator may input a desired concentration of the anesthetic agent to be delivered to the patient. A corresponding valve position of one or more valves of the anesthesia machine (e.g., a position of one or more bypass valves, as described above) may be empirically determined and stored in a predetermined lookup table or function in a memory of the controller. For example, the controller may receive the desired concentration of the anesthetic agent via the input device and may determine an amount of opening of the one or more valves corresponding to the desired concentration of the anesthetic agent based on the lookup table, with the input being the concentration of the anesthetic agent and the output being the valve position of the one or more valves. The controller may transmit an electrical signal to an actuator of the one or more valves in order to adjust each of the one or more valves to the corresponding output valve position. In some examples, the controller may compare the desired flow rate of gases to a measured flow rate of gases, such as measured by the inspiratory flow sensor, for example.

Further, the adjustments described above may be facilitated at least in part based on output from the respiratory gas module 108. The respiratory gas module 108 may be configured to measure various parameters of the gases exiting the anesthetic vaporizer 114 and/or being provided to the patient. For example, the respiratory gas module 108 may measure the concentrations of carbon dioxide, nitrous oxide, and the anesthetic agent provided to the patient. Further, the respiratory gas module 108 may measure respiration rate, minimum alveolar concentration, patient oxygen, and/or other parameters. The output from the respiratory gas module 108 may be displayed the anesthesia display device 104 and/or the patient monitoring display device 106 and/or used by the controller 140 to provide closed-loop feedback control of the amount of anesthesia provided to the patient.

The controller 140 is shown in FIG. 1 for illustrative purposes, and it is to be understood that the controller 140 may be located in various locations within, around, and/or remote from the anesthesia machine 100. As an example, the controller 140 may include multiple devices/modules that may be distributed throughout the anesthesia machine 100. As such, the controller 140 may include a plurality of controllers at various locations within the anesthesia machine 100. As another example, additionally or alternatively, the controller 140 may include one or more devices/modules that are external to the anesthesia machine 100, located proximate to (e.g., in the patient care location 101) or remote from (e.g., a remote server) the anesthesia machine 100. In each example, the multiple devices/modules may be communicatively coupled through wired and/or wireless connections.

As mentioned above, gas delivered to the anesthesia machine 100 via the first pipeline gas supply outlet 150 and the second pipeline gas supply outlet 152 may originate at a central medical gas distribution system. The central medical gas distribution system may be located in a same facility (e.g., a healthcare facility) as the anesthesia machine 100 but in a different area of the facility, for example. Therefore, the first pipeline gas supply outlet 150 and the second pipeline gas supply outlet 152 may provide the anesthesia machine 100 with medical gas from a remote location within the facility. For example, a pipeline network may carry the medical gas from the central medical gas distribution system to the first pipeline gas supply outlet 150 and the second pipeline gas supply outlet 152, which may serve as terminal outlets for the medical gas at a point of use (e.g., the patient care location 101). In one embodiment, the pipeline network is comprised of copper pipes. The first pipeline gas supply outlet 150 and the second pipeline gas supply outlet 152 may be color-coded based on the medical gas delivered and labeled with the medical gas name Further, the first pipeline gas supply outlet 150 and the second pipeline gas supply outlet 152 may each include self-sealing sockets that accept a gas-specific plug to couple the tubing to the corresponding pipeline gas connector (e.g., first pipeline connector 146 or second pipeline connector 147, respectively), thereby reducing an incidence of an incorrect gas being connected to a particular pipeline gas supply connector.

The central medical gas distribution system may include various equipment, including (but not limited to) gas-holding cylinders and/or tanks, gas manifolds (e.g., coupled to the gas-holding cylinders and/or tanks), air compressors, vacuum pumps, generators, and concentrators. For example, some types of medical gas, such as nitrogen, nitrous oxide, and carbon dioxide, may be purchased from an outside supplier in pre-filled cylinders. The pre-filled cylinders may be coupled to a manifold that automatically switches from an empty cylinder to a full cylinder (e.g., in response to a pressure of the cylinder decreasing below a threshold pressure that indicates that the cylinder is empty) in order to supply a constant stream of gas. Thus, the pipeline gas supply for such gases may include the pre-filled cylinders, the manifold, and the piping network coupled to the manifold, as well as various valves (e.g., shut-off valves), filters, sensors, and the pipeline gas supply outlet. Other types of medical gas, such as air, may be generated on-site by the central medical gas distribution system. For example, ambient air may be compressed by an air compressor of the central medical gas distribution system, dried via an air dryer, and stored in air tanks and/or cylinders (e.g., via a filling system). Thus, in such an example, the pipeline gas supply may also include the air compressor and the air dryer.

In some examples, oxygen also may be generated on-site. For example, a portion of the compressed and dried air (which is approximately 78% nitrogen, 21% oxygen, and 1% argon and other gases) may be distributed to an oxygen generator that separates the oxygen component of the air from the other components. The oxygen may be concentrated via an oxygen concentrator to produce gas that is approximately 92-93% oxygen (e.g., greater than 90% oxygen). Thus, the pipeline gas supply for oxygen generated via an oxygen concentrator may further include the oxygen generator and the oxygen concentrator. In other embodiments, oxygen may be purchased from an outside supplier in pre-filled cylinders and/or tanks instead of generated on-site. In such an embodiment, the gas in the pre-filled cylinders and/or tanks may be approximately 100% oxygen.

Thus, the central medical gas distribution system may include a pipeline gas supply for each of the various medical gases, each pipeline gas supply including equipment for storing, distributing, and (in some examples) generating the corresponding medical gas. In particular, the gases generated on-site (e.g., air and optionally oxygen) may be exposed to more potential sources of contamination compared with gases sourced from pre-filled cylinders and/or tanks. For example, water is a common contaminant of medical air that may be introduced via inadequate drying via the air dryers (such as from using an undersized dryer or due to dryer saturation, for example), via degraded air compressor components, or via degradation of other central medical gas distribution system components. Oil, another potential contaminant, may be introduced via the compressor, such as when a non-medical grade compressor is used or compressor degradation occurs. The oil may also break down into various liquid and gaseous hydrocarbons. Further, particulate debris may be introduced to the pipeline network from sand, dirt, solder, flux, metal filings, vermin, cement, desiccant dust, fibers, lint, etc. As another example, biological contaminants, such as viruses, bacteria, fungus (e.g., mold spores), and pollen may be introduced to the medical gas supply within the pipeline network and/or within the anesthesia machine 100. For example, oil-water aerosols may cover the inner surfaces of the pipeline network and act as a growth medium for micro-organisms. In extensive pipeline networks, blind loops and other locations suitable for bacteria proliferation may occur. Additional potential liquid and gaseous contaminants may include cleaning chemical residues, plasticizer out-gassing, halogenated solvents, methane, carbon monoxide, nitrogen oxide, hydrogen fluoride, hydrogen sulfate, carbon dioxide, chlorine, and halogenated refrigerants.

While the central medical gas distribution system may include various monitors for detecting medical gas contamination and alarms for alerting localized personnel of the contamination, the alarms may be limited to the central medical gas distribution system location. Thus, contamination that occurs downstream of the central medical gas distribution system may not be detected. Further, the alarms may not actively prevent further delivery of the contaminated gas to downstream equipment, such as the anesthesia machine 100. For example, the operator of the anesthesia machine 100 may be unaware of the alarms at the central medical gas distribution system location and may continue to operate the anesthesia machine with the contaminated gas. As a result, the contaminated gas may degrade components of the anesthesia machine 100 and may be supplied to the patient.

Therefore, FIG. 2 shows an embodiment of a gas quality monitor 200 that may be used to detect contaminants within a gas. The gas quality monitor 200 includes a housing 202 that encloses a measurement passage 204 and a plurality of sensors. The measurement passage 204 provides a gas flow path through the gas quality monitor. The embodiment shown in FIG. 2 includes a first sensor 206, a second sensor 208, a third sensor 210, and a fourth sensor 212. However, other embodiments may include a different number of sensors, such as more than four sensors or fewer than four sensors. Each sensor may be a different type of sensor configured to measure (e.g., detect or sense) a different quantity or component within the gas flowing through the measurement passage 204. The quantities/components may include, but are not limited to, an amount (or concentration) of total volatile organic compounds (tVOCs) in the gas, an amount carbon dioxide (CO₂) in the gas, an amount of particulate matter, a temperature of the gas, and a humidity of the gas, as will be elaborated below. Thus, each sensor is configured to obtain a different type of measurement. Further, each sensor (e.g., the first sensor 206, the second sensor 208, the third sensor 210, and the fourth sensor 212) is electronically connected to a data acquisition device 214. The data acquisition device 214 may be an embedded system, a system-on-chip, a microcontroller, or another electronic device included within the housing 202 and configured to receive measurements from the plurality of sensors and output raw and/or processed measurement data to a remote network, as will be elaborated below with respect to FIG. 3. For example, the data acquisition device 214 may include wireless communication technology, such as Wi-Fi and/or Bluetooth, to wirelessly communicate with other controllers/networks that are external to the housing 202. The gas quality monitor 200 may be an Internet of Things (IoT) device, for example.

As will be elaborated below with respect to FIGS. 3-7, the gas quality monitor 200 may be coupled in various locations within or external to a medical gas flow device, such as the anesthesia machine 100 shown in FIG. 1, such as at or near a gas inlet port or outlet port of the medical gas flow device. Further, more than one gas quality monitor 200 may be fluidically coupled to or within the medical gas flow device. The gas enters the gas quality monitor 200 at an inlet coupling 216, flows through the measurement passage 204, and exits the gas quality monitor 200 at an outlet coupling 218. The gas received at the inlet coupling 216 originates from a gas source, such as a pipeline gas supply, and the gas exiting at the outlet coupling 218 may directly or indirectly flow to a patient. As one example, the inlet coupling 216 may form a gas-tight seal with a conduit providing the gas from the gas source to the medical gas flow device, such as tubing 154 of FIG. 1, so that the gas flows from the gas source into the gas quality monitor 200 without escaping, and the outlet coupling 218 may form a gas-tight seal with an inlet port of the medical gas flow device (e.g., the first pipeline connector 146 of FIG. 1) so that the gas flows from the gas quality monitor 200 to the medical gas flow device (and onto the patient) without escaping. As another example, the inlet coupling 216 may form a gas-tight seal with an outlet port of the medical gas flow device (e.g., inspiratory port 118 of FIG. 1) so that the gas flows from the medical gas flow device into the gas quality monitor 200 without escaping, and the outlet coupling 218 may form a gas-tight seal with a patient delivery passage (e.g., inspiratory gas passage 121 of FIG. 1) so that the gas flows from the gas quality monitor 200 to the patient without escaping.

The first sensor 206, the second sensor 208, the third sensor 210, and the fourth sensor 212 are coupled to the measurement passage 204 to measure various qualities of the gas flowing therein. As one example, the first sensor 206 may be a humidity sensor, the second sensor 208 may be a tVOC (or hydrocarbon) sensor, the third sensor 210 may be a particulate matter sensor, and the fourth sensor 212 may be a carbon dioxide sensor. However, other combinations of gas quality sensors may be used that ensure that the gas flowing through the gas quality monitor 200 is clean, dry, and of an expected composition. In the present example, the humidity sensor, configured to detect water vapor, may output a signal to the data acquisition device 214 indicating an amount (or dew point) of water vapor in the gas flowing through the measurement passage 204; the tVOC sensor, configured to detect organic compounds (including grease and oil), may output a signal to the data acquisition device 214 indicating an amount (or concentration) of organic compounds in the gas supplied flowing through the measurement passage 204; the particulate matter sensor, configured to detect organic and inorganic particles suspended in the gas, may output a signal to the data acquisition device 214 indicating an amount (or concentration) of particulate matter in the gas flowing through the measurement passage 204; and the carbon dioxide may output a signal to the data acquisition device 214 indicating an amount (or concentration) of carbon dioxide in the gas flowing through the measurement passage 204.

As an illustrative example, medical air is often generated via a compressor and gas drying system, as described above. Insufficient gas drying, which may result in water vapor in the air, may be detected via the humidity sensor. As one example, the humidity sensor may be configured to measure both a temperature and a moisture (e.g., water vapor) content of the air supplied from the gas source to determine a relative humidity of the air (e.g., a ratio of the measured moisture in the air to the maximum possible amount of moisture at the measured temperature, which may be expressed as a percentage). As another example, aerobic organisms, including many bacteria and fungi, release carbon dioxide through cellular respiration. Therefore, the measurement made by the carbon dioxide sensor may be used to detect carbon dioxide emitted by biological contaminants. The tVOC sensor may indicate contamination by oil, such as oil from the compressor or delivery pipes, or bacterial metabolites (e.g., acetone, ethanol, or acetic acid). The particulate matter sensor may indicate particulate contamination, such as where the air is not sufficiently filtered and/or is contaminated downstream of the filters. Water vapor, oil, bacterial/fungal growth, and particulate contamination of the air may degrade components of the medical gas flow device, for example. Further, delivery of a clean, high quality medical gas to the patient is desired. Therefore, in response to any of the measured qualities being outside of a corresponding allowable range, potential contamination may be indicated, as will be further described below with respect to FIGS. 8A and 8B.

Continuing to FIG. 3, a schematic depiction of a first embodiment of a medical gas quality monitoring system 300 is shown. In the embodiment shown, the medical gas quality monitoring system 300 is integrated within the anesthesia machine 100 introduced in FIG. 1. As such, components previously introduced in FIG. 1 are numbered the same and will not be reintroduced. However, in other embodiments, the medical gas quality monitoring system 300 may be coupled to another type of medical gas flow device, such as a free-standing ventilator or an incubator. Further, for illustrative clarity, some of the components of anesthesia machine 100 introduced in FIG. 1 are not shown in FIG. 3, although it may be understood that those components may be present.

The medical gas quality monitoring system 300 includes a first inlet gas quality monitor 302 and a second inlet gas quality monitor 304, each coupled within the housing 102 of the anesthesia machine at a gas inlet to the anesthesia machine 100. Thus, the medical gas quality monitoring system 300 is internal to the anesthesia machine 100. In particular, the first inlet gas quality monitor 302 is coupled within a first gas flow passage 306, which flows a first gas received from the first pipeline gas supply outlet 150 via the tubing 154 and the first pipeline connector 146. Together, the first pipeline gas supply outlet 150, the tubing 154, the first gas flow passage 306, the first inlet gas quality monitor 302, and associated connectors provide a gas flow path for the first gas through anesthesia machine 100. The second inlet gas quality monitor 304 is coupled within a second gas flow passage 308, which flows a second gas received from the second pipeline gas supply outlet 152 via the tubing 156 and the second pipeline connector 147. The second pipeline gas supply outlet 152, the tubing 156, the second gas flow passage 308, the second inlet gas quality monitor 304, and associated connectors provide a gas flow path for the second gas through the anesthesia machine 100.

In the embodiment shown in FIG. 3, the first gas flow passage 306 includes a first flow control valve 312 positioned therein, and the second gas flow passage 308 includes a second flow control valve 314 positioned therein. The first flow control valve 312 may be adjusted between a fully closed position and a fully open position to vary a relative amount of the first gas that flows from the first pipeline gas supply outlet 150 to the inspiratory port 118. Similarly, the second flow control valve 314 may be adjusted between a fully closed position and a fully open position to vary a relative amount of the second gas that flows from the second pipeline gas supply outlet 152 to the inspiratory port 118. Further, the first gas flow passage 306 joins with the second gas flow passage 308 at a junction 310 that is downstream of each of the first flow control valve 312 and the second flow control valve 314. Thus, the first gas and the second gas converge and mix at and downstream of the junction 310 before flowing to the inspiratory port 118 and to the patient via the inspiratory gas passage 121. It may be understood that the arrangement of the gas flow passages 306 and 308 is illustrative, and additional or alternative gas flow passages may be present between the gas inlets (e.g., at the first pipeline connector 146 and the second pipeline connector 147) and the gas outlet (e.g., at the inspiratory port 118) and/or between other gas supplies and the gas outlet.

The first inlet gas quality monitor 302 and the second inlet gas quality monitor 304 each include a plurality of sensors, a measurement passage, and a data acquisition device, as elaborated above with respect to FIG. 2. Thus, the first inlet gas quality monitor 302 and the second inlet gas quality monitor 304 may both have the configuration described above with reference to the gas quality monitor 200 of FIG. 2. The first inlet gas quality monitor 302 and the second inlet gas quality monitor 304 are each communicatively coupled to a remote network 305. The remote network 305 may be a Cloud computing network, for example, and is also communicatively coupled to the controller 140 of the anesthesia machine 100 and a portable user interface 315. The portable user interface 315 may be configured to both output information to a user (e.g., via a display screen and/or speakers) and receive inputs from the user (e.g., via a touchscreen, a touchpad, a stylus, a mouse, and/or a keyboard). For example, the portable user interface 315 may be a tablet, a smartphone, a smartwatch, or a laptop and may be located remote from or in a same room as the anesthesia machine 100.

The sensors included in the first inlet gas quality monitor 302 may measure a plurality of qualities in the first gas as it flows through the first inlet gas quality monitor 302, and the measurements may be wirelessly transmitted from the first inlet gas quality monitor 302 to the remote network 305, such as over a wireless personal area network (e.g., WPAN), Bluetooth, or another wireless communication technology. Similarly, the sensors included in the second inlet gas quality monitor 304 may measure a plurality of qualities of the second gas as it flows through the second inlet gas quality monitor 304, and the measurements may be wirelessly transmitted from the second inlet gas quality monitor 304 to the remote network 305. The remote network 305 may further communicate the measurements, which may include raw and/or processed measurement data, to the portable user interface 315 and/or the controller 140.

Because of the positioning of first inlet gas quality monitor 302, the first inlet gas quality monitor 302 measures only the first gas. Further, the measurements obtained by the first inlet gas quality monitor 302 may be used to detect contaminants introduced into the first gas upstream of the first inlet gas quality monitor 302, such as within the first pipeline connector 146, the tubing 154, the first pipeline gas supply outlet 150, and/or the gas source coupled thereto. Similarly, the second inlet gas quality monitor 304 is positioned to measure only the second gas. The measurements obtained by the second inlet gas quality monitor 304 may be used to detect contaminants introduced into the second gas upstream of the second inlet gas quality monitor 304, such as within the second pipeline connector 147, the tubing 156, the second pipeline gas supply outlet 152, and/or the gas source coupled thereto. Thus, the medical gas quality monitoring system 300 includes monitoring the gas input into the anesthesia machine 100, and may not detect contaminants introduced into either the first gas or the second gas at a location upstream of the first inlet gas quality monitor 302 and the second inlet gas quality monitor 304.

Next, FIG. 4 schematically shows a second embodiment of a medical gas quality monitoring system 400. The medical gas quality monitoring system 400 is substantially identical to the medical gas quality monitoring system 300 introduced in FIG. 3 except for the differences described below. As such, like components previously introduced in FIGS. 1 and 3 are numbered the same and function as previously described with respect to FIGS. 1 and 3.

In the embodiment shown, the medical gas quality monitoring system 400 is external to the anesthesia machine 100. That is, in contrast to the medical gas quality monitoring system 300 of FIG. 3, the first inlet gas quality monitor 302 and the second inlet gas quality monitor 304 are positioned outside of the housing 102 of the anesthesia machine 100 in the medical gas quality monitoring system 400. Specifically, in the embodiment shown in FIG. 4, the first inlet gas quality monitor 302 is coupled within tubing 154, between the first pipeline gas supply outlet 150 and the first pipeline connector 146, and the second inlet gas quality monitor 304 is coupled within tubing 156, between the second pipeline gas supply outlet 152 and the second pipeline connector 147. As such, the medical gas quality monitoring system 400 may be more easily installed into existing anesthesia machines than the medical gas quality monitoring system 300 of FIG. 3. However, because of the external location of the first inlet gas quality monitor 302 and the second inlet gas quality monitor 304 in the medical gas quality monitoring system 400 shown in FIG. 4, the medical gas quality monitoring system 400 may not detect contaminants originating at the first pipeline connector 146 and the second pipeline connector 147.

FIG. 5 schematically shows a third embodiment of a medical gas quality monitoring system 500. The medical gas quality monitoring system 500 is substantially identical to the medical gas quality monitoring system 300 introduced in FIG. 3 (and the medical gas quality monitoring system 400 introduced in FIG. 4) except for the differences described below. As such, like components previously introduced in FIGS. 1 and 3 are numbered the same and function as previously described with respect to FIGS. 1 and 3.

The medical gas quality monitoring system 500 includes an outlet gas quality monitor 502 coupled within the housing 102 of the anesthesia machine 100 at a gas outlet of the anesthesia machine 100. Thus, the medical gas quality monitoring system 500 is internal to the anesthesia machine 100. The outlet gas quality monitor 502 may be substantially identical to the first inlet gas quality monitor 302 and the second inlet gas quality monitor 304 introduced in FIG. 3 except for its positioning at the outlet.

In the embodiment shown, the outlet gas quality monitor 502 is coupled within the second gas flow passage 308, downstream of the junction 310 with the first gas flow passage 306 and upstream of the inspiratory port 118. Thus, the outlet gas quality monitor 502 is positioned downstream of where the first gas and the second gas converge, and the sensors of the outlet gas quality monitor 502 measure the resultant mixture of the first gas and the second gas. As described above with respect to FIG. 3, the measurements may be wirelessly transmitted from the outlet gas quality monitor 502 to the remote network 305, and the remote network 305 may further communicate the measurements to the portable user interface 315 and/or the controller 140.

Because of the position of the outlet gas quality monitor 502, the measurements output by the outlet gas quality monitor 502 may be used to detect contaminants introduced into both the first gas and the second gas upstream of the outlet gas quality monitor 502, such as within the first pipeline connector 146, the second pipeline connector 147, the tubing 154, the tubing 156, the first pipeline gas supply outlet 150 and/or the gas source coupled thereto, the second pipeline gas supply outlet 152 and/or the gas source coupled thereto, the first gas flow passage 306, the second gas flow passage 308, the first flow control valve 312, and/or the second flow control valve 314. Thus, the medical gas quality monitoring system 500 is positioned to detect contaminants in the gas input into the anesthesia machine 100 and as well as contaminants introduced into the gas within the anesthesia machine 100, prior to its output at the inspiratory port 118. Further, the medical gas quality monitoring system 500 may not detect contaminants introduced into the gas at a location upstream of the outlet gas quality monitor 502, such as at the inspiratory port 118 or within the inspiratory gas passage 121.

Next, FIG. 6 schematically shows a fourth embodiment of a medical gas quality monitoring system 600. The medical gas quality monitoring system 600 is substantially identical to the medical gas quality monitoring system 500 introduced in FIG. 5 (and the medical gas quality monitoring system 300 of FIG. 3 and the medical gas quality monitoring system 400 of FIG. 4) except for the differences described below. As such, like components previously introduced in FIGS. 1 and 3-5 are numbered the same and function as previously described with respect to FIGS. 1 and 3-5.

The medical gas quality monitoring system 600 includes the outlet gas quality monitor 502 coupled outside of the housing 102 of the anesthesia machine 100 at a gas outlet of the anesthesia machine 100. Thus, in contrast to the medical gas quality monitoring system 500 of FIG. 5, the medical gas quality monitoring system 600 is external to the anesthesia machine 100. In the embodiment shown in FIG. 6, the outlet gas quality monitor 502 is coupled within the inspiratory gas passage 121, downstream of the inspiratory port 118. Because of the external location of the outlet gas quality monitor 502 in the medical gas quality monitoring system 600 shown in FIG. 6, the medical gas quality monitoring system 600 may additionally detect contaminants introduced into the gas flowing through the inspiratory gas passage 121 at the inspiratory port 118. Further, the medical gas quality monitoring system 600 may be more easily installed into existing anesthesia machines than the medical gas quality monitoring system 500 of FIG. 5.

FIG. 7 schematically shows a fifth embodiment of a medical gas quality monitoring system 700. The medical gas quality monitoring system 700 is substantially identical to the medical gas quality monitoring systems introduced in FIGS. 3-6, particularly the medical gas quality monitoring system 300 of FIG. 3 and the medical gas quality monitoring system 500 of FIG. 5, except for the differences described below. As such, like components previously introduced in FIGS. 1 and 3-6 are numbered the same and function as previously described with respect to FIGS. 1 and 3-6.

The medical gas quality monitoring system 700 includes the first inlet gas quality monitor 302, the second inlet gas quality monitor 304, and the outlet gas quality monitor 502. Because the medical gas quality monitoring system 700 includes both the inlet gas quality monitors 302 and 304 and the outlet gas quality monitor 502, comparing measurements from the inlet gas quality monitors 302 and 304 and the outlet gas quality monitor 502 may enable contaminant sources to be localized, as will be elaborated below with respect to FIGS. 8A and 8B. As an illustrative example, when the outlet gas quality monitor 502 outputs measurements indicative of bacterial contamination and both of the first inlet gas quality monitor 302 and the second inlet gas quality monitor 304 do not, it may be inferred that the source of the bacterial contamination is within the anesthesia machine 100, downstream of the inlet gas quality monitors 302 and 304 and upstream of the outlet gas quality monitor 502. As another illustrative example, when the outlet gas quality monitor 502 outputs measurements indicative of particulate contamination and the first inlet gas quality monitor 302 also outputs measurements indicative of particulate contamination (and the second inlet gas quality monitor 304 does not), it may be inferred that the source of the particulate contamination is upstream of the first inlet gas quality monitor 302.

Note that although the medical gas quality monitoring system 700 is integrated within the anesthesia machine 100, other embodiments are also possible. For example, one or more or each of the first inlet gas quality monitor 302, the second inlet gas quality monitor 304, and the outlet gas quality monitor 502 may be coupled outside of the housing 102, such as shown in FIGS. 3 and 5. As one example, the medical gas monitoring system may include the first inlet gas quality monitor 302 and the second inlet gas quality monitor 304 positioned within the housing 102 and the outlet gas quality monitor 502 positioned external to the housing 102. As another example, the medical gas monitoring system may include the first inlet gas quality monitor 302 and the second inlet gas quality monitor 304 positioned outside of the housing 102 and the outlet gas quality monitor 502 positioned inside of the housing 102. As still another example, the medical gas monitoring system may include the first inlet gas quality monitor 302 positioned within the housing 102, the second inlet gas quality monitor 304 positioned external to the housing 102, and the outlet gas quality monitor 502 positioned external to the housing 102. Thus, the embodiments shown in FIGS. 3-7 are provided by way of example, and other embodiments of the medical gas monitoring system may include different numbers, locations, and relative positions (e.g., with respect to the housing of the anesthesia machine) of the gas quality monitors.

Turning now to FIGS. 8A and 8B, an example method 800 is shown for operating a medical gas quality monitoring system, such as any of the medical gas quality monitoring systems described above with respect to FIGS. 3-7, to monitor a gas provided to and/or from a medical gas flow device (e.g., the anesthesia machine 100 introduced in FIG. 1). The medical gas quality monitoring system includes at least one gas quality monitor, and each of the at least one gas quality monitor may have the configuration of the gas quality monitor 200 shown in FIG. 2. Method 800 may be carried out by one or more controllers, such as the controller 140 of FIG. 1 and/or the data acquisition device 214 of FIG. 2, according to instructions stored in a memory of the controller and in conjunction with one or more sensors (e.g., the first sensor 206, the second sensor 208, the third sensor 210, and the fourth sensor 212 of FIG. 2) and actuators (e.g., first flow control valve 312 and second flow control valve 314 of FIGS. 3-7).

As one example, method 800 may be executed while the medical gas flow device is operated to provide medical gas to a patient, enabling real-time quality monitoring of the provided medical gas. As used herein, the term “real-time” refers to obtaining, processing, and/or outputting information without intended delay. Additionally or alternatively, method 800 may be executed during a power-on-self-test (POST) of the medial gas flow device, while the medical gas flow device is not providing the medical gas to a patient. As still another example, method 800 may be strategically executed before and after execution of a disinfection routine or other sterilization procedure, as will be elaborated below. For simplicity, method 800 is described with respect to monitoring a single medical gas; however, it may be understood that method 800 may be performed in parallel for each of a plurality of gases supplied to the medical gas flow device.

At 802, method 800 includes obtaining gas measurements via sensors the gas quality monitor(s). As one example, the medical gas quality monitoring system may include only an inlet gas quality monitor coupled to an inlet of the medical gas flow device and no gas quality monitor coupled to an outlet of the medical gas flow device, such as the medical gas quality monitoring system 300 of FIG. 3 or the medical gas quality monitoring system 400 of FIG. 4. As another example, the medical gas quality monitoring system may include only an outlet gas quality monitor coupled to the outlet of the medical gas flow device and no gas quality monitor coupled to the inlet, such as the medical gas quality monitoring system 500 of FIG. 5 or the medical gas quality monitoring system 600 of FIG. 6. As still another example, the medical gas quality monitoring system may include both the inlet gas quality monitor and the outlet gas quality monitor. Thus, the sensors may be positioned to measure qualities of the gas as it is supplied to the medical gas flow device (e.g., at the inlet) and/or after the gas has flowed through the medical gas flow device (e.g., at the outlet). Further, the gas may be any medical gas, such as medical air, oxygen, nitrogen, nitrous oxide, carbon dioxide, etc. and may be supplied from a gas source. The gas source may include one or more pre-filled cylinders, manifolds, pipes, valves, filters, sensors, compressors, dryers, and/or concentrators, as elaborated above. In particular, components of the gas source may be housed at a location that is remote from the medical gas quality monitoring system and the medical gas flow device, and the gas may be delivered to the medical gas flow device via a network of conduits (e.g., pipes and tubing).

The sensors may obtain the gas measurements as the gas flows through a measurement passage of each included gas quality monitor (e.g., measurement passage 204). As described above with respect to FIG. 2, the obtained gas measurements may be for any measurable aspect of interest, including (but not limited to) a water vapor content (e.g., a concentration, dew point, or relative humidity), a tVOC or hydrocarbon content, a particulate content, and a carbon dioxide content, with the particular aspect measured by each sensor based on the type of sensor used (e.g., a humidity sensor for measuring the water vapor content of the gas, a tVOC sensor for measuring the tVOC content of the gas, a particulate matter sensor for measuring the particulate content of the gas, and a carbon dioxide sensor for measuring the carbon dioxide content of the gas). Further, in embodiments of the medical gas quality monitoring system that include additional sensor(s), the additional sensor(s) may each measure an additional aspect (e.g., oxygen content measured by an oxygen sensor). In this way, multiple different aspects of the gas are measured at 802.

At 804, method 800 includes determining a gas quality index of the gas based on the received gas measurements. For example, the controller may combine the currently received individual sensor measurements to generate a single, easy to understand gas quality index value. As one example, the controller may input each gas measurement into a look-up table, algorithm, or model, which may output the corresponding gas quality index for the input measurements. For example, the gas quality index may rate or score an overall relative quality of the gas on a standardized scale, with lower values corresponding to lower gas quality and higher values corresponding to higher gas qualities. As an example, higher water vapor content, tVOC content, particulate content, and carbon dioxide content measurements may decrease the gas quality index. Further, the standardized scale may be divided into descriptive grade ranges to aid interpretation of the gas quality index. As an illustrative example using a scale out of 100, gas quality index values falling between 95 and 100 may be given an “excellent quality” grade, gas quality index values between 90 and 94 may be given a “good quality” grade, gas quality index values between 80 and 89 may be given a “moderate quality” grade, gas quality index values between 70 and 79 may be given a “poor quality” grade, and gas quality index values between 1 and 69 may be given a “very poor quality” grade, although other grades and grade ranges may be used. Further, the controller may update the gas quality index as it changes based on the current measurements received from the sensors.

In some examples where more than one gas quality monitor is coupled to the gas flow, the controller may determine separate gas quality index values from the measurements received from each gas quality monitor. As one example, the gas quality index values may be combined, such as averaged. As another example, the controller may adjust a first gas quality index value (e.g., determined based on the measurements received from the inlet gas quality monitor) using a second gas quality index value (e.g., determined based on the measurements received from the outlet gas quality monitor. In other examples, the controller may determine a single gas quality index value using the measurements received from each gas quality monitor, such as by inputting each gas measurement into the look-up table, algorithm, or model, as described above.

At 806, method 800 includes outputting the gas quality index. As an example, the gas quality index may be output to a user interface (e.g., the portable user interface 315 of FIGS. 3-7), such as to a display screen of the user interface or via an audible message. As an example, both the gas quality index value and the grade may be output to the display. In some examples, each of the measured water vapor content, tVOC content, particulate content, and carbon dioxide content may be output in addition to the gas quality index.

At 807, method 800 includes determining if the gas quality index is less than a threshold. The threshold refers to a pre-determined gas quality index value above which the gas is expected to be clean, dry, and of an expected composition. For example, it may be inferred that the gas is not contaminated when the gas quality index is greater than the threshold, as contamination would result in measurements that would decrease the gas quality index below the threshold. As one example, the threshold may be a lower bound of the “excellent quality” grade range (e.g., 95 in the example given above at 804). As another example, the threshold may be a lower bound of the “good quality” range (e.g., 90 in the example given above at 804).

If the gas quality index is not less than the threshold (e.g., the gas quality index is greater than or equal to the threshold), method 800 proceeds to 809 and includes storing the sensor measurements and the determined gas quality index in a log. For example, the log may organize the sensor measurements according to time. Thus, the newly obtained sensor measurements (e.g., current sensor measurement) may be stored with previously obtained sensors measurements (e.g., previous sensor measurements) that may be utilized as aggregate data, as will be elaborated below with respect to 812. Further, by storing the determined gas quality index in the log, a user, such as a facilities manager or healthcare professional, may be able to track the gas quality index over time to identify trends in gas quality. The log may be stored in a memory, which may be a local memory of the controller or a remote memory accessed via a network (e.g., remote network 305 of FIG. 3). Method 800 may then end.

If instead the gas quality index is less than the threshold, method 800 proceeds to 808 (see FIG. 8B) and includes determining if moisture is present. For example, it may be determined that moisture is present responsive to the measured water vapor content being greater than or equal to a threshold water vapor content. The threshold water vapor content may be a pre-calibrated, non-zero value stored in memory and correspond to a water vapor content at or above which the gas is improperly dried. As an example, water vapor may condense within various conduits, couplings, valves, etc., and act as a growth medium for micro-organisms, such as bacteria and mold. As another example, when the water vapor content is higher than the threshold water vapor content, unintentionally humidified gas may be provided to a patient. Further, the threshold water vapor content may vary based on the gas being measured and/or the measurement location (e.g., the inlet or the outlet of the medical gas flow device).

If moisture is present (e.g., the measured water vapor content is at or above the threshold water vapor content), method 800 proceeds to 810 and includes outputting a moisture alert. The moisture alert may be output via the user interface and may include a visual message or symbol signifying that moisture has been detected in the gas. Additionally or alternatively, the moisture alert may include an audible message or alarm sound. As mentioned above, detecting moisture in the gas may correspond with generally lower gas quality index values.

At 812, method 800 includes evaluating the gas for contamination based on aggregate data obtained over time. Further, the method may proceed to 812 responsive to moisture not being detected at 808 (e.g., the measured water vapor content is less than the threshold water vapor content). Thus, whether or not moisture is present in the gas, the gas is evaluated for additional biological and non-biological contamination. The aggregate data may include not only the current sensor measurements, but previous measurements obtained from each sensor over a pre-determined duration, such as stored in the log. As one example, the pre-determined duration may be a calibrated amount of time over which a biological contaminant may establish a colony large enough to produce a measureable metabolite, for example. The metabolite may be carbon dioxide, an alcohol (e.g., ethanol), a ketone (e.g., acetone), and/or a carboxylic acid (e.g., acetic acid), for example.

The controller may evaluate whether the measurements obtained by each sensor increase, decrease, or remain the same over the pre-determined duration, including a rate of increase/decrease and a timing of the increase/decrease of one sensor measurement relative to the others. Additionally or alternatively, the controller may compare the aggregate data to a plurality of contamination models stored in memory (e.g., non-transitory memory), each of the plurality of contamination models corresponding to a different type of potential contaminant (e.g., biological, particulate, or chemical), a combination of potential contaminants, or no contaminants, and determine which of the plurality of contamination models best fits (e.g., matches) the aggregate data. As one example, each of the plurality of contamination models may include prophetic measurements, measurement trends, etc. for each of the different sensors. Thus, by comparing the aggregate data to the plurality of contamination models, the controller may more accurately identify a type of contamination than when each individual sensor measurement is compared to a threshold, for example. However, in some examples, each of the plurality of models may additionally or alternatively include respective thresholds for each sensor measurement, as will be elaborated below. Further, the respective threshold for each sensor measurement may be the same or different between each model. As an illustrative example, a threshold tVOC content may be higher for a chemical contamination model and lower for a biological contamination model.

Further, when more than one gas quality monitor is included, the aggregate data for each gas quality monitor may be analyzed separately, at least in some examples. In such examples, the aggregate data from the input gas quality monitor may be compared against the plurality of models, and the aggregate data from the output gas quality monitor may be compared against the plurality of models independently from the aggregate data from the input gas quality monitor. In this way, the best fitting model for the aggregate data from the (upstream) input gas quality monitor may be different than the best fitting model of the (downstream) outlet gas quality monitor.

At 814, method 800 includes determining if biological contamination is present, such as when the aggregate data matches a model including biological contaminants. As one example, the particulate content may increase prior to the tVOC and carbon dioxide content of the gas increasing when bacteria are present. Further, the particulate content may increase more gradually when bacteria are present than when non-biological particulate contamination is present. Further still, the particulate content may be comprised of differently sized particles depending on the type of biological contamination present. For example, viruses are smaller than bacteria, and some types of mold spores are larger than bacteria. Relative particle sizes of different biological and non-biological contaminants will be elaborated below with respect to FIG. 9. Further, when the gas is evaluated by more than one gas quality monitor, it may be determined that biological contamination is present responsive to the aggregate data from at least one of the gas quality monitors best fitting a model including biological contaminants.

In an example, if a gas delivery system is outfitted with both the inlet gas quality monitor and the outlet gas quality monitor, the differences in measurements of particulate count (e.g., a spectrum of particulate measurements indicating bacteria and mold), tVOCs, and elevated CO₂ would act as indicators of biological contamination within the gas delivery system. Likewise, if multiple gas quality monitoring systems are networked together (e.g., via the remote network 305 of FIG. 3, for example) and the same gas quality index measurements are made across a fleet of gas delivery systems, if a particular system's measurement is an outlier in the dataset, it may be inferred that potential biological contamination is present within that gas delivery system.

If biological contamination is present, method 800 proceeds to 816 and includes outputting a biological contamination alert. The biological contamination alert may be output via the user interface and may include a visual message or symbol signifying that biological contamination has been detected in the gas. Additionally or alternatively, the biological contamination alert may include an audible message or alarm sound. Further, detecting biological contamination in the gas may correspond with generally lower gas quality index values, as the particulate content, tVOC content, and carbon dioxide content may be higher.

At 818, method 800 includes inferring a location of the contamination based on coupling location(s) of the gas quality monitor(s). In order to be detected, the biological contaminant particles and metabolites may flow directionally with the gas to be delivered to the measurement passage, and it may be inferred that the location of the contamination is generally upstream of the gas quality monitor. Therefore, if the gas quality monitoring system includes only the inlet gas quality monitor, then the location of the contamination may be within the delivery network from the gas source. If the gas quality monitoring system includes only the outlet gas quality monitor, then the location of the contamination may be within the delivery network from the gas source or within the medical gas flow device.

As mentioned above, if the gas quality monitoring system includes both an inlet gas quality monitor and an outlet gas quality monitor, then the contamination may be more precisely localized based on whether one or both of the gas quality monitors detects the presence of biological contamination. For example, if the upstream inlet gas quality monitor does not detect biological contamination and the downstream outlet gas quality monitor does detect biological contamination, then it may be inferred that the location of the contamination is within the medical gas flow device, between the inlet gas quality monitor and the outlet gas quality monitor. As another example, if the inlet gas quality monitor and the outlet gas quality monitor both detect biological contamination, then it may be inferred that the location is upstream of the inlet gas quality monitor, such as exterior to the medical gas flow device. The inferred location may be output to the user interface device in order to aid additional cleaning and inspection procedures, for example.

At 820, method 800 includes performing a disinfection routine. For example, even if the inferred location of the contamination is external to the medical gas flow device (e.g., upstream of the medical gas flow device), the biological contaminants may be spread throughout downstream conduits, valves and connectors, for example. Therefore, performing the disinfection routine may help prevent additional colonies from forming within the medical gas flow device.

Performing the disinfection routine may include activating a UVGI system, such as the UVGI system 160 shown in FIG. 1, to irradiate internal gas flow components (e.g., conduits, valves, and connectors) with UV (e.g., UV-C) light. The UVGI system may be activated for a pre-determined amount of time that is efficacious for inactivating irradiated microorganisms, such as 30 minutes. In some examples, performing the disinfection routine may further include flushing the gas flow passages of the medical gas flow device with a high concentration of oxygen, such as greater than 60% oxygen, and the UV light may induce formation of reactive oxygen species that kills or inactivates the microbes. As one example, a gas flow control valve (e.g., the first flow control valve 312 or the second flow control valve 314 of FIGS. 3-7) positioned in an oxygen gas flow passage may be fully opened to enable the high concentration of oxygen to flow through at least portions of the medical gas flow device.

Returning to 814, if biological contamination is not present, such as when the aggregate data does match a model including biological contaminants, method 800 proceeds to 822 and includes determining if particulate contamination is present. As one example, particulate contamination may be determined to be present responsive to the model best fitting the aggregate data including particulate contaminants As one example, particulate contamination may be present when the particulate content increases without a subsequent increase in carbon dioxide and/or tVOCs. Further, particulate contamination may be present when the particulate content increases at a faster rate than when biological contamination is present. Further still, the particulate content may be comprised of differently sized particles depending on the type of particulate contamination present. For example, suspended atmospheric dust is smaller than settling dust, and settling dust is smaller than heavy, as will be elaborated below with respect to FIG. 9. Further, when the gas is evaluated by more than one gas quality monitor, it may be determined that particulate contamination is present responsive to the aggregate data from at least one of the gas quality monitors best fitting a model including particulate contaminants.

Further, method 800 may include evaluating the gas for particulate contamination even if biological contamination is present, such as in addition to initiating a response to the biological contamination described with respect to 816-820. For example, the aggregate data may fit a model including a combination of particulate contaminants and biological contaminants.

In an example, if a gas delivery system is outfitted with both the inlet gas quality monitor and the outlet gas quality monitor, the differences in measurements of particulate count may indicate potential particulate contamination within the gas delivery system. Likewise, if multiple gas quality monitoring systems are networked together and the same gas quality index measurements are made across a fleet of gas delivery systems, if a particular gas delivery system's measurement is an outlier in the data, particulate contamination may be present within that gas delivery system.

As another example, additionally or alternatively, the particulate matter sensor may include a particulate counter, which can measure both mass and size fraction (such as PM1, PM2.5, PM10 and coarse, which is greater than PM10), and the particulate counter may be used to define a spectrum of particulate contaminants present. The measurement spectrum/quantization of PM1 and PM2.5 versus PM10 and coarse may be further used to separate measurements of potential mold/bacteria from larger non-biologic particulates such as dust, scale, lint, etc. As will be elaborated below with respect to FIG. 9, PM10 measurements may be used to define particles less than 10 μm in size (coarser fine dust and organic particles), and coarse measurements may be used to define coarse particles, which are 10 μm or larger (for example, visible coarse dust, fibers, and large organic particles). If PM1 and PM2.5 particulate measurements are combined with tVOC and/or CO₂ measurements indicative of organic metabolites, it may be determined that a possible biological contaminant (mold/bacteria) is present, whereas elevated PM10 and coarse measurements, and/or the absence of tVOC and/or CO₂ measurements indicative of organic metabolites, may indicate the presence of particulate contamination.

If particulate contamination is present, method 800 proceeds to 824 and includes outputting a particulate contamination alert. The particulate contamination alert may be output via the user interface and may include a visual message or symbol signifying that particulate contamination has been detected in the gas. Additionally or alternatively, the particulate contamination alert may include an audible message or alarm sound. Further, detecting particulate contamination in the gas may correspond with generally lower gas quality index values, as the particulate content may be higher.

If particulate contamination is not present, such as when the best fitting model to the aggregate data does not include particulate contaminants, method 800 proceeds to 826 and includes determining if chemical contamination is present. As one example, chemical contamination may be determined to be present responsive to the model best fitting the aggregate data including chemical contaminants. As one example, chemical contamination may be present when the tVOC content is above a tVOC threshold while the carbon dioxide content remains below a carbon dioxide threshold. Together, the tVOC threshold and the carbon dioxide threshold may be pre-determined thresholds calibrated to distinguish tVOC and carbon dioxide increases from biological contaminants with high tVOC content due to cleaning solvents, chemicals emitted from plastics, etc. As another example, chemical contamination may be present when the tVOC content is above the tVOC threshold while the particulate content is less than a pre-determined particulate threshold, the particulate threshold calibrated to distinguish chemical contamination from biological contamination. Thus, it may be determined that chemical contamination is present responsive to a lack of significant particulate and/or CO₂ measurements (e.g., below respective pre-determined thresholds) and elevated tVOC measurements (e.g., above the tVOC threshold). Further, when the gas is evaluated by more than one gas quality monitor, it may be determined that chemical contamination is present responsive to the aggregate data from at least one of the gas quality monitors best fitting a model including chemical contaminants.

Further, method 800 may include evaluating the gas for chemical contamination even if particulate contamination is present, such as in addition to outputting the particulate contamination alert at 824. For example, the aggregate data may fit a model including a combination of particulate contaminants and chemical contaminants, biological contaminants and chemical contaminants, or a combination of all three. As another example, the aggregate data may fit a model including only chemical contamination (e.g., and not particulate contamination or biological contamination).

If chemical contamination is not present, such as when chemical contamination is not included in the model that best fits the aggregate data, method 800 proceeds to 832 and includes storing the sensor measurements, the determined gas quality index, and any output alert(s) in the log. Thus, the newly obtained sensor measurements may become part of the aggregate data. Further, by storing the determined gas quality index and any output alert(s) in the log, the user may be able to identify trends in a contamination occurrence and track facilities-wide gas quality degradation and contamination concerns. As an example, the particulate contamination alert may occur more frequently after maintenance at the central gas distribution facility. As another example, the moisture alert and the biological contamination alert may occur more frequently in gas sources that use a compressor. These trends may aid the user in updating maintenance and cleaning protocols, for example, or in deciding which gas vendor to purchase from.

Following 832, method 800 ends. For example, method 800 may be repeated at a pre-determined frequency. As another example, method 800 may be repeated responsive to a detected change in the output of one of the sensors while the medical gas flow device is being operated (e.g., powered on, with gas flowing through the medical gas flow device). As such, the updated gas measurements may be used to update the gas quality index and output any alerts regarding the moisture, biological contamination, particulate contamination, and chemical contamination accordingly.

As still another example, method 800 may be repeated after executing the disinfection routine at 820 in order to evaluate and/or quantify an effectiveness of the UVGI system, for example. As an example, the controller may compare the medical gas quality index prior to performing the disinfection routine and the medical gas quality index after performing the disinfection routine to quantify a change (e.g., increase) in the medical gas quality index value resulting from performing the disinfection routine. As another example, the controller may directly compare markers of biological contaminants, such as tVOC and/or carbon dioxide measurements, immediately before and immediately after performing the disinfection routine to quantify the effectiveness of the disinfection routine, and thus, the UVGI system. Such information may be output to a user (e.g., via the display), stored in the log, and/or communicated to the remote network.

Returning to 826, if chemical contamination is present, such as when the best fitting model to the aggregate data includes chemical contaminants, method 800 proceeds to 828 and includes outputting a chemical contamination alert. The chemical contamination alert may be output via the user interface and may include a visual message or symbol signifying that chemical contamination has been detected in the gas. Additionally or alternatively, the chemical contamination alert may include an audible message or alarm sound. Further, detecting chemical contamination in the gas may correspond with generally lower gas quality index values, as the tVOC content may be higher.

At 830, method 800 optionally includes performing a flushing routine. For example, if the medical gas quality monitoring system includes the inlet gas quality monitor and the outlet gas quality monitor, the flushing routine may be performed responsive to the outlet gas quality monitor detecting chemical contamination and the inlet gas quality monitor not detecting chemical contamination, indicating that the chemical contamination is within the medical gas flow device and not originating at the gas source. The flushing routine may be performed while the medical gas flow device is not currently being used to provide medical gas to a patient and may include flowing the gas through the medical gas flow device at a high flow rate for a pre-determined flushing duration. As an example, the flow rate may be higher than that used for delivering the medical gas to a patient. As another example, the flushing routine may include actuating the flow control valve to a fully open position in order to increase the gas flow through the medical gas flow device. By flushing gas through the medical gas flow device, lingering cleaning chemicals may be evaporated and forced through, for example. Method 800 may then proceed to 832, as described above.

In this way, a quality of a medical gas flowing provided to and/or from a medical gas flow device may be monitored. By alerting a user, such as a clinician or a facility manager, to gas quality degradation, delivery of low quality gases to a patient may be avoided. Further, by performing a disinfection routine responsive to biological contamination being present and performing a flushing routine responsive to chemical contamination being present, the medical gas quality may be increased. Further still, an effectiveness of the disinfection routine and/or the flushing routine may be evaluated by repeating method 800 after performing the routine(s).

Further, although the example method 800 includes evaluating the medical gas for moisture and other contaminants responsive to a gas quality index being less than a threshold, in other examples, the method may include evaluating the medical gas for moisture and other contaminants even when the gas quality index is greater than or equal to the threshold.

Turning now to FIG. 9, an example chart 900 comparing the size distribution of potential particulate contaminants, including biological contaminants and non-biological contaminants, is shown. The biological contaminants are represented by diagonally shaded oblong shapes, and the non-biological contaminants are represented by unshaded oblong shapes. The size, in micrometers (um), increases horizontally from left to right and is shown as a logarithmic scale on the top of chart 900. Further, different particle size ranges are shown at the bottom of chart 900, including PM1 (e.g., particulate matter having a diameter of 1 μm or less), PM2.5 (e.g., particulate matter a diameter of 2.5 μm or less), PM10 (e.g., particulate matter a diameter of 10 μm or less), and coarse (e.g., particulate matter a diameter greater than 10 μm). A particulate matter sensor of a gas quality monitoring system, such as the third sensor 210 shown in FIG. 2, may measure a particle size and density of particulates within a gas flow, and the gas quality monitoring system may determine a gas quality index of the gas flow based in part on the measured size and density. Smaller, PM1 and PM2.5 particles may reduce the gas quality index to a greater degree than larger, PM10 and course particles, for example.

The biological contaminants include viruses 902, bacteria 904, mold spores 906, and pollen 908. The non-biological contaminants include suspended atmospheric dust 912, settling dust 914, and heavy dust 916. The viruses 902 are included in the PM1 and PM2.5 size ranges. The bacteria 904 are primarily in the PM10 range, although some of the bacteria 904 are small enough to be detected in the PM1 and PM2.5 ranges or large enough to be detected in the coarse range. The mold spores 906 may be detected in the PM10 or coarse measurements. The pollen 908 may be detected in the coarse measurements. Thus, the viruses 902 and the bacteria 904 may have a greater impact the gas quality index than the mold spores 906 and the pollen 908.

The suspended atmospheric dust 912 is detectable in the PM1 and PM2.5 ranges. The settling dust 914 spans the detection ranges and may be detected in the PM1, PM2.5, PM10, and coarse measurement ranges. In contrast, the heavy dust 916 is only detected in the coarse measurement range. Thus, the suspended atmospheric dust 912 and the settling dust 914 may have a greater impact on the gas quality index than the heavy dust 916.

Further, because of size overlaps, the biological contaminants and the non-biological contaminants may not be distinguishable in particulate matter sensor measurements. For example, the suspended atmospheric dust 912 may not be distinguishable from the viruses 902 because both are found within the PM1 range. As another example, the heavy dust 916 may be indistinguishable from the pollen 908 due to their overlapping measurement range. Therefore, the gas quality monitoring system may include additional sensors that may help distinguish at least some of the biological contaminants from the non-biological contaminants, as described above with respect to FIGS. 8A and 8B.

Next, FIGS. 10-12 show example timelines for detecting contamination in a medical gas via a medical gas quality monitoring system. The medical gas quality monitoring system includes a first, upstream gas quality monitor positioned at an inlet of a medical gas delivery system (e.g., the anesthesia machine 100 introduced in FIG. 1) and a second, downstream gas quality monitor positioned at an outlet of the medical gas delivery system, such as the example medical gas quality monitoring system shown in FIG. 7. Each of the first and second gas quality monitors include a humidity sensor, a tVOC sensor, a particulate matter sensor, and a carbon dioxide sensor. Thus, each of FIGS. 10-12 shows an output of an upstream humidity sensor (included in the first gas quality monitor) in a plot 1002, an output of a downstream humidity sensor (included in the second gas quality monitor) in a dashed plot 1004, an output of an upstream tVOC sensor in a plot 1006, an output of a downstream tVOC sensor in a dashed plot 1008, an output of an upstream particulate matter sensor in a plot 1010, an output of a downstream particulate matter sensor in a dashed plot 1012, an output of an upstream carbon dioxide sensor in a plot 1014, and an output of a downstream carbon dioxide sensor in a dashed plot 1016.

For all of the above, the horizontal axis represents time, with time increasing along the horizontal axis from left to right. The vertical axis represents a magnitude of each sensor output, with the magnitude increasing up the vertical axis from bottom to top. Note that although each sensor output is shown as a continuous graph, the sensor output may not be received continuously over time. For example, the sensor measurements may be received at a pre-determined frequency and/or while the medical gas flow device is operated. For example, each plot may include distinct sensor measurements obtained across multiple operations of the medical gas flow device and stored in memory. Further, the particulate matter sensor output is shown as a single output corresponding to a total amount of particulate matter detected and is not divided into different measurement size ranges (such as PM1, PM2.5, etc.). However, in other examples, the particulate matter sensor may include multiple outputs, each output corresponding to a different measurement size range.

Further still, the examples shown in FIGS. 10-12 include respective contamination detection thresholds for each sensor output, including a humidity sensor output threshold (dashed line 1001), a tVOC sensor output threshold (dashed line 1005), a particulate matter sensor output threshold (dashed line 1009), and a carbon dioxide sensor output threshold (dashed line 1013), although in other examples, only some sensor outputs may have an associated threshold (e.g., the humidity sensor output threshold), or none of the sensor outputs may have an associated threshold for detecting contamination. For example, a controller may automatically compare the sensor outputs to a contamination model to determine if contamination is present without comparing the output of each sensor to a threshold, as elaborated above with respect to FIGS. 8A and 8B. Thus, the timelines shown in FIGS. 10-12 represent one example of how the information received from the different sensors may be combined to detect contamination in of a medial gas.

Turning first to FIG. 10, a first prophetic example timeline 1000 of detecting a distinguishing a type of contamination in the medical gas is shown. Prior to time t1, the upstream humidity sensor output (plot 1002) and the downstream humidity sensor output (dashed plot 1004) are substantially the same, indicating that there is not a significant difference in the humidity of the gas at the inlet of the medical gas flow device and at the outlet of the medical gas flow device. Further, the upstream humidity sensor output (plot 1002) and the downstream humidity sensor output (dashed plot 1004) are both below the humidity sensor output threshold (dashed line 1001), indicating that moisture is not detected in the gas. Similarly, the upstream tVOC sensor output (plot 1006), the downstream tVOC sensor output (dashed plot 1008), the upstream particulate matter sensor output (plot 1010), the downstream particulate matter sensor output (dashed plot 1012), the upstream carbon dioxide sensor output (plot 1014), and the downstream carbon dioxide sensor output (dashed plot 1016) all remain below their respective thresholds prior to time t1.

At time t1, the upstream humidity sensor output (plot 1002) and the downstream humidity sensor output (dashed plot 1004) both reach the humidity sensor output threshold (dashed line 1001). In response, a controller determines that moisture is present in the medical gas and outputs a moisture alert. Further, because both of the upstream humidity sensor output (plot 1002) and the downstream humidity sensor output (dashed plot 1004) reach the humidity sensor output threshold (dashed line 1001) and remain substantially the same, the controller may infer that the source of the moisture is upstream of the first gas quality monitor and not between the first gas quality monitor and the second gas quality monitor. Further, at time t1, the upstream tVOC sensor output (plot 1006), the downstream tVOC sensor output (dashed plot 1008), the upstream particulate matter sensor output (plot 1010), the downstream particulate matter sensor output (dashed plot 1012), the upstream carbon dioxide sensor output (plot 1014), and the downstream carbon dioxide sensor output (dashed plot 1016) all remain below their respective thresholds, with the upstream and downstream measurements of each sensor type remaining substantially the same.

Between time t1 and time t2, the downstream particulate matter sensor output (dashed plot 1012) increases relative to the upstream particulate matter sensor output (plot 1010). At time t2, the downstream particulate matter sensor output (dashed plot 1012) reaches the particulate matter sensor output threshold (dashed line 1009) while the upstream particulate matter sensor output (plot 1006) remains below the particulate matter sensor output threshold. In response, the controller infers that particulate matter is originating at a source located between the first gas quality monitor and the second gas quality monitor, such as within the medical gas flow device. The controller compares the outputs of each sensor obtained over a pre-determined time period to a particulate contamination model. However, in the example of timeline 1000, the combined outputs of each sensor do not match the particulate contamination model due to the gradual increase in the downstream particulate matter sensor output (dashed plot 1012). Therefore, the controller continues monitoring the output of each sensor.

Between time t2 and time t3, the downstream tVOC sensor output (dashed plot 1008) increases relative to the upstream tVOC sensor output (plot 1006). Further, the downstream carbon dioxide sensor output (dashed plot 1016) increases relative to the upstream carbon dioxide sensor output (plot 1014). At time t3, the downstream tVOC sensor output (dashed plot 1008) increases above the tVOC sensor output threshold (dashed line 1005) while the upstream tVOC sensor output (plot 1006) remains below the tVOC sensor output threshold. In response, the controller infers that volatile organic compounds are originating at a source located between the first gas quality monitor and the second gas quality monitor, such as within the medical gas flow device. Further, in the example shown in FIG. 10, the controller evaluates the outputs of each sensor obtained over the pre-determined time period against a biological contamination model responsive to the tVOC sensor output threshold being surpassed. Because the outputs include the downstream particulate matter sensor output (dashed plot 1012) increasing prior to the downstream carbon dioxide sensor output (dashed plot 1016) and the downstream tVOC sensor output (dashed plot 1008) increasing, the controller determines that biological contamination is present. Further, the controller infers that the source of the biological contamination is between the first gas quality monitor and the second gas quality monitor, within the medical gas flow device.

Responsive to detecting the biological contamination within the medical gas flow device, the controller executes a disinfection routine at time t4. In particular, the controller activates a UVGI system (e.g., UVGI system 160 of FIG. 1) to irradiate internal components of the medical gas flow device with UV-C light. As a result, after time t4, the downstream tVOC sensor output (dashed plot 1008) and the downstream carbon dioxide sensor output (dashed plot 1016) both quickly decrease, as the biological contaminants cease to generate dioxide and volatile organic compound metabolites prior to the disinfection routine. The output of the downstream particulate matter sensor (dashed plot 1012) also begins to decrease, but decreases more slowly as the killed biological contaminants may take longer to clear from the medical gas flow device.

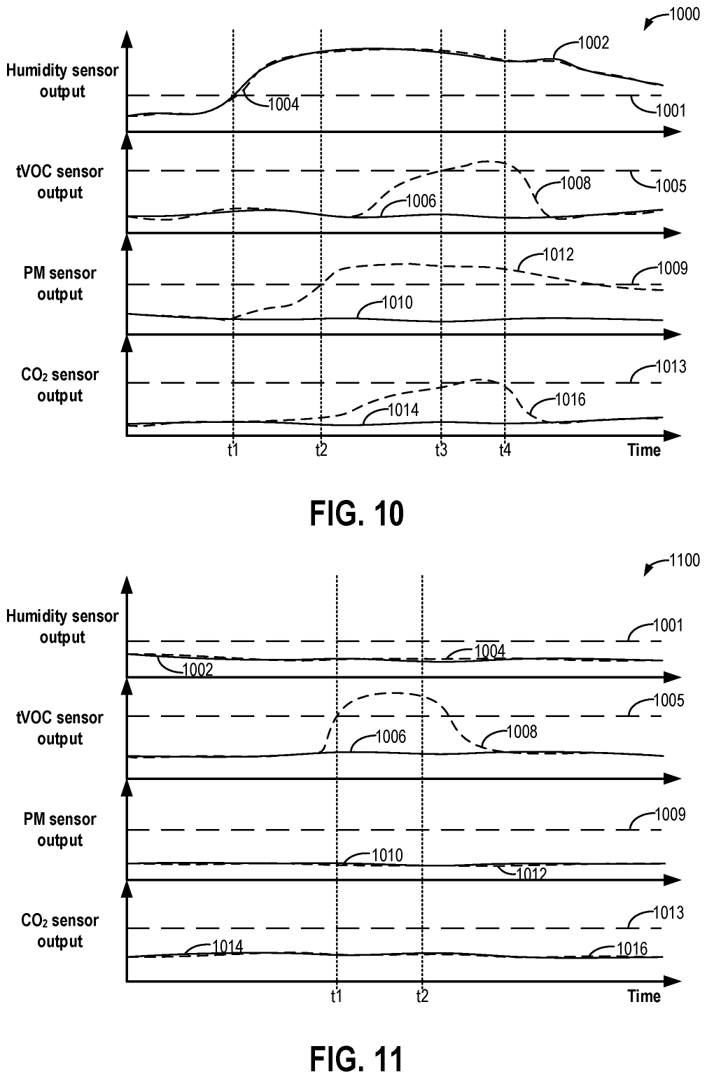

Turning next to FIG. 11, a second prophetic example timeline 1100 for detecting contamination in the medical gas is shown. Prior to time t1, the upstream humidity sensor output (plot 1002) and the downstream humidity sensor output (dashed plot 1004) are substantially the same, indicating that there is not a significant difference in the humidity of the gas at the inlet of the medical gas flow device and at the outlet of the medical gas flow device. Further, the upstream humidity sensor output (plot 1002) and the downstream humidity sensor output (dashed plot 1004) are both below the humidity sensor output threshold (dashed line 1001), indicating that moisture is not detected in the gas. Similarly, the upstream particulate matter sensor output (plot 1010) and the downstream particulate matter sensor output (dashed plot 1012) are substantially the same and remain below the particulate matter sensor output (dashed line 1009), and the upstream carbon dioxide sensor output (plot 1014) and the downstream carbon dioxide sensor output (dashed plot 1016) are substantially the same and remain below the carbon dioxide sensor output (dashed line 1013). However, just before time t1, the downstream tVOC sensor output (dashed plot 1008) increases relative to the upstream tVOC sensor output (plot 1006) and reaches the tVOC sensor output threshold (dashed line 1005) at time t1.

In response to the downstream tVOC sensor output reaching the tVOC sensor output threshold, the controller compares the output of each sensor obtained over the pre-determined duration to a plurality of models, including a chemical contamination model. In the example of timeline 1100, the chemical contamination model fits the output of each sensor obtained over the pre-determined duration. In particular, because the upstream tVOC sensor output (plot 1006) remains below the tVOC sensor output threshold, the controller determines that the chemical contamination is present between the inlet and the outlet of the medical gas flow device.

In response to determining that chemical contamination is present within the medical gas flow device, at time t2, the controller performs a flushing routine to deliver the medical gas flow device at a high flow rate for a pre-determined flushing duration in order to evaporate and/or push the chemical contamination out of the medical gas flow device while it is not being used to provide the medical gas to the patient. As a result, after time t2, the downstream tVOC sensor output (dashed plot 1008) decreases and becomes substantially equal to the upstream tVOC sensor output (plot 1006).

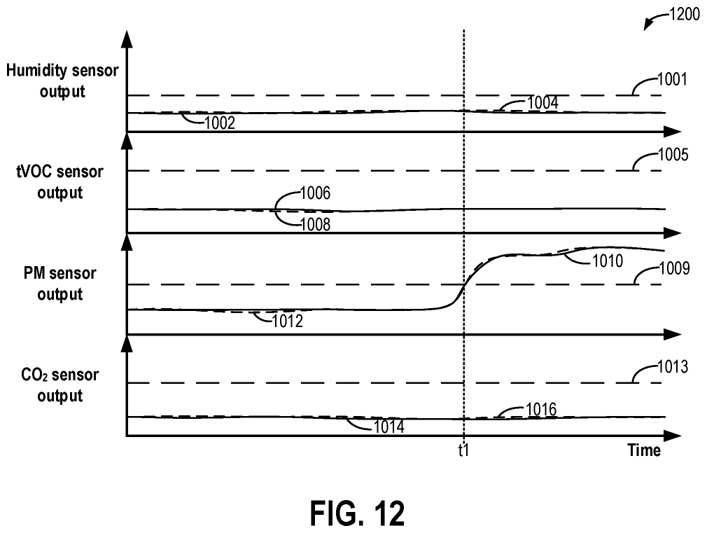

FIG. 12 shows a third prophetic example timeline 1200 for detecting contamination in the medical gas. Prior to time t1, the upstream humidity sensor output (plot 1002) and the downstream humidity sensor output (dashed plot 1004) are substantially the same, indicating that there is not a significant difference in the humidity of the gas at the inlet of the medical gas flow device and at the outlet of the medical gas flow device. Further, the upstream humidity sensor output (plot 1002) and the downstream humidity sensor output (dashed plot 1004) are both below the humidity sensor output threshold (dashed line 1001), indicating that moisture is not detected in the gas. Similarly, the upstream tVOC sensor output (plot 1006) and the downstream tVOC sensor output (plot 1008) are substantially the same and remain below the tVOC sensor output threshold (dashed line 1005), and the upstream carbon dioxide sensor output (plot 1014) and the downstream carbon dioxide sensor output (dashed plot 1016) are substantially the same and remain below the carbon dioxide sensor output (dashed line 1013). The upstream particulate matter sensor output (plot 1010) and the downstream particulate matter sensor output (dashed plot 1012) are also substantially the same. However, both the upstream particulate matter sensor output (plot 1010) and the downstream particulate matter sensor output (dashed plot 1012) begin to increase in a synchronized fashion prior to time t1.

At time t1, both the upstream particulate matter sensor output (plot 1010) and the downstream particulate matter sensor output (dashed plot 1012) reach the particulate matter sensor output threshold (dashed line 1009). In response, the controller compares the output of each sensor obtained over the pre-determined duration to a plurality of models, including particulate contamination model. In the example of timeline 1200, the particulate contamination model fits the output of each sensor obtained over the pre-determined duration. In particular, the particulate contamination model is the best fitting model because the upstream tVOC sensor output (plot 1006) and the downstream tVOC sensor output (plot 1008) are substantially the same and remain below the tVOC sensor output threshold (dashed line 1005), and the upstream carbon dioxide sensor output (plot 1014) and the downstream carbon dioxide sensor output (dashed plot 1016) are substantially the same and remain below the carbon dioxide sensor output (dashed line 1013). Thus, the controller determines that particulate contamination is present. Further, because both the upstream particulate matter sensor output (plot 1010) and the downstream particulate matter sensor output (dashed plot 1012) are greater than the particulate matter sensor output threshold (dashed line 1009), the controller determines that a source of the particulate contamination is present upstream of the first gas quality monitor and introduced to the medical gas prior to the medical gas reaching the medical gas flow device.

Thus, the systems and methods described herein provide for a smart medical gas delivery module, enabling an uninterrupted supply of clean, dry medical gas of an expected composition to be delivered to a gas delivery system without human intervention. As a result, equipment and patient exposure to a contaminated or wrong medical gas is limited, thereby decreasing gas delivery system degradation and potentially increasing patient safety. By decreasing gas delivery system degradation, an amount of time that the gas delivery system is out of service is decreased and maintenance costs are decreased. Further, an accuracy of a gas mixture delivered by the gas delivery system to a patient may be increased. Overall, gas delivery system operator satisfaction may be increased.

A technical effect of monitoring a quality of a medical gas supplied from a medical gas pipeline to a gas delivery system upstream of an inlet to the gas delivery system and automatically switching to an alternative gas supply if the quality is outside of an allowable range is that degradation of the gas delivery system is decreased while the gas delivery system receives an uninterrupted supply of gas.

In one embodiment, a method for a medical gas quality monitoring system comprises: obtaining measurements of a medical gas via a plurality of sensors, the plurality of sensors including at least one of a humidity sensor, a particulate matter sensor, a carbon dioxide sensor, and a total volatile organic compound (tVOC) sensor; determining a gas quality index of the medical gas based on the obtained measurements; and outputting the determined gas quality index. In examples, the method further comprises evaluating the medical gas for contamination based on the obtained measurements and previous measurements obtained over time; responsive to the contamination not being present, storing the obtained measurements with the previous measurements obtained over time; and responsive to the contamination being present, storing the obtained measurements with the previous measurements obtained over time and outputting a contamination alert to the display.

In one example, evaluating the medical gas for the contamination based on the obtained measurements and the previous measurements obtained over time is responsive to the determined gas quality index being less than a threshold gas quality index.

In some examples, evaluating the medical gas for the contamination based on the obtained measurements and the previous measurements obtained over time includes evaluating the medical gas for one or more of biological contamination, non-biological particulate contamination, and chemical contamination. In an example, evaluating the medical gas for one or more of the biological contamination, the non-biological particulate contamination, and the chemical contamination comprises: identifying a best fitting model to the obtained measurements and the previous measurements obtained over time from a plurality of models, each of the plurality of models including prophetic measurement from the plurality of sensors for one or a combination of the biological contamination, the non-biological particulate contamination, and the chemical contamination; indicating the biological contamination is present responsive to the best fitting model including the biological contamination; indicating the non-biological particulate contamination is present responsive to the best fitting model including the non-biological particulate contamination; and indicating the chemical contamination is present responsive to the best fitting model including the chemical contamination.

In an example, each of the humidity sensor, the particulate matter sensor, the carbon dioxide sensor, and the tVOC sensor are included in the plurality of sensors, and evaluating the medical gas for contamination based on the obtained measurements and the previous measurements obtained over time comprises: evaluating the medical gas for biological contamination by combining the obtained measurements and the previous measurements obtained over time from the particulate matter sensor, the carbon dioxide sensor, and the tVOC sensor; evaluating the medical gas for non-biological particulate contamination by combining the obtained measurements and the previous measurements obtained over time from the particulate matter sensor, the carbon dioxide sensor, and the tVOC sensor; evaluating the medical gas for chemical contamination by combining the obtained measurements and the previous measurements obtained over time from the particulate matter sensor and the tVOC sensor; and evaluating the medical gas for water vapor contamination based on the obtained measurements and the previous measurements obtained over time from the humidity sensor.

In an example, the obtained measurements and the previous measurements obtained over time comprise aggregate data, and evaluating the medical gas for contamination based on the obtained measurements and the previous measurements obtained over time comprises: outputting a biological contamination alert responsive to the aggregate data matching a biological contamination model; outputting a particulate contamination alert responsive to the aggregate data matching a particulate contamination model; and outputting a chemical contamination alert responsive to the aggregate data matching a chemical contamination model.

As one example, the humidity sensor is included in the plurality of sensors, and the method further comprises outputting a moisture alert responsive to a water vapor content measured by the humidity sensor increasing above a threshold water vapor content.

In an example, outputting the determined gas quality index includes wirelessly transmitting the determined gas quality index to a display of a portable user interface via a remote network.

An embodiment of a medical gas quality monitoring system comprises: a first gas quality monitor coupled at a first position in a gas flow path, the first gas quality monitor including a plurality of sensors positioned to measure quantities within a medical gas flowing through the gas flow path at the first position; a user interface including a display; and a controller including instructions stored in non-transitory memory that, when executed, cause the controller to: receive measurements from the plurality of sensors of the first gas quality monitor; determine a gas quality index value using the received measurements; output the determined gas quality index value to the display; and output a contamination alert responsive to the gas quality index value being less than a threshold.

In an example, the first position is internal to a housing of a medical gas flow device positioned at a patient care location.

In another example, the first position is external to a housing of a medical gas flow device positioned at a patient care location.

In examples, the first position is at an inlet to a medical gas flow device positioned at a patient care location. In some examples, the medical gas quality monitoring system further comprises a second gas quality monitor coupled at an outlet of the medical gas flow device, the second gas quality monitor including a second plurality of sensors positioned to measure quantities within the medical gas flowing through the gas flow path at the outlet, and wherein the controller includes further instructions stored in non-transitory memory that, when executed, cause the controller to: receive measurements from the second plurality of sensors of the second gas quality monitor; and adjust the gas quality index value using the received measurements from the second gas quality monitor.

As an example, the first position is at an outlet of a medical gas flow device, upstream of a gas passage configured to couple the outlet to a patient breathing circuit, and external to a housing of the medical gas flow device.

As another example, the first position is at an outlet of a medical gas flow device, upstream of a gas passage configured to couple the outlet to a patient breathing circuit, and internal to a housing of the medical gas flow device.

In an embodiment, a system comprises: a gas source; a gas flow device including a patient delivery passage; a delivery network fluidically coupling the gas source to the gas flow device; a medical gas quality monitoring system including at least one gas quality monitor, each of the at least one gas quality monitor including each of a plurality of different types of sensors positioned to measure a gas flow originating from the gas source at a location upstream of the patient delivery passage; and a controller including instructions stored in non-transitory memory that, when executed, cause the controller to: monitor a quality of the gas flow in real-time based on current measurements received from each of the plurality of different types of sensors; and evaluate the gas flow for potential contamination in real-time based on the current measurements and previous measurements received from one or more or each of the plurality of different types of sensors.

In examples, the plurality of different types of sensors include a humidity sensor, a volatile organic compound sensor, a particulate matter sensor, and a carbon dioxide sensor. In an example, the controller further includes a plurality of contamination models stored in non-transitory memory, and to evaluate the gas flow for potential contamination, the controller includes further instructions stored in non-transitory memory that, when executed, cause the controller to: evaluate the gas flow for potential biological contamination by comparing the current measurements and the previous measurements received from the volatile organic compound sensor, the particulate matter sensor, and the carbon dioxide sensor to a biological contamination model of the plurality of contamination models; evaluate the gas flow for potential particulate contamination by comparing the current measurements and the previous measurements received from the volatile organic compound sensor, the particulate matter sensor, and the carbon dioxide sensor to a particulate contamination model of the plurality of contamination models; and evaluate the gas flow for potential chemical contamination by comparing the current measurements and the previous measurements received from the volatile organic compound sensor and the particulate matter sensor to a chemical contamination model of the plurality of contamination models. In another example, the controller includes further instructions stored in non-transitory memory that, when executed, cause the controller to: evaluate the gas flow for moisture by comparing the current measurement received from the humidity sensor to a threshold.

In another representation, a method for a medical gas quality monitoring system comprises: evaluating a medical gas flowing through a gas delivery system for biological contamination based on measurements obtained from a plurality of sensors, each sensor of the plurality of sensors positioned to measure a quantity within the medical gas; and responsive to detecting biological contamination, performing a disinfection routine. In the preceding example, additionally or optionally, performing the disinfection routine includes activating an ultraviolet germicidal irradiation (UVGI) system for a threshold duration, the UVGI system positioned to irradiate components of the gas delivery system with UV-C light. In one or both of the preceding examples, the method additionally or optionally further comprises, immediately after performing the disinfection routine, evaluating an effectiveness of the disinfection routine. In any or all of the preceding examples, additionally or optionally, evaluating the effectiveness of the disinfection routine includes quantifying a reduction in biological contaminants based on the measurements obtained from the plurality of sensors prior to performing the disinfection routine relative to measurements obtained from the plurality of sensors immediately after performing the disinfection routine. In any or all of the preceding examples, additionally or optionally, the plurality of sensors include each of a carbon dioxide sensor, a particulate matter sensor, and a volatile organic compound sensor, and evaluating the medical gas flowing through the gas delivery system for biological contamination based on measurements obtained from the plurality of sensors includes indicating biological contamination is present responsive to detecting an increase in an a particulate measurement obtained from the particulate matter sensor followed by an increase in one or more of a carbon dioxide measurement obtained from the carbon dioxide sensor and a volatile organic compound measurement obtained from the volatile organic compound sensor.

As used herein, an element or step recited in the singular and proceeded with the word “a” or “an” should be understood as not excluding plural of said elements or steps, unless such exclusion is explicitly stated. Furthermore, references to “one embodiment” of the present invention are not intended to be interpreted as excluding the existence of additional embodiments that also incorporate the recited features. Moreover, unless explicitly stated to the contrary, embodiments “comprising,” “including,” or “having” an element or a plurality of elements having a particular property may include additional such elements not having that property. The terms “including” and “in which” are used as the plain-language equivalents of the respective terms “comprising” and “wherein.” Moreover, the terms “first,” “second,” and “third,” etc. are used merely as labels, and are not intended to impose numerical requirements or a particular positional order on their objects.

This written description uses examples to disclose the invention, including the best mode, and also to enable a person of ordinary skill in the relevant art to practice the invention, including making and using any devices or systems and performing any incorporated methods. The patentable scope of the invention is defined by the claims, and may include other examples that occur to those of ordinary skill in the art. Such other examples are intended to be within the scope of the claims if they have structural elements that do not differ from the literal language of the claims, or if they include equivalent structural elements with insubstantial differences from the literal languages of the claims.

1. A method for a medical gas quality monitoring system, comprising:
obtaining measurements of a medical gas via a plurality of sensors, the plurality of sensors including at least one of a humidity sensor, a particulate matter sensor, a carbon dioxide sensor, and a total volatile organic compound (tVOC) sensor;
determining a gas quality index of the medical gas based on the obtained measurements; and
outputting the determined gas quality index.

2. The method of claim 1, further comprising:
evaluating the medical gas for contamination based on the obtained measurements and previous measurements obtained over time;
responsive to the contamination not being present, storing the obtained measurements with the previous measurements obtained over time; and
responsive to the contamination being present, storing the obtained measurements with the previous measurements obtained over time and outputting a contamination alert to the display.

3. The method of claim 2, wherein evaluating the medical gas for the contamination based on the obtained measurements and the previous measurements obtained over time is responsive to the determined gas quality index being less than a threshold gas quality index.

4. The method of claim 2, wherein evaluating the medical gas for the contamination based on the obtained measurements and the previous measurements obtained over time includes evaluating the medical gas for one or more of biological contamination, non-biological particulate contamination, and chemical contamination.

5. The method of claim 4, wherein evaluating the medical gas for one or more of the biological contamination, the non-biological particulate contamination, and the chemical contamination comprises:
identifying a best fitting model to the obtained measurements and the previous measurements obtained over time from a plurality of models, each of the plurality of models including prophetic measurement from the plurality of sensors for one or a combination of the biological contamination, the non-biological particulate contamination, and the chemical contamination;
indicating the biological contamination is present responsive to the best fitting model including the biological contamination;
indicating the non-biological particulate contamination is present responsive to the best fitting model including the non-biological particulate contamination; and
indicating the chemical contamination is present responsive to the best fitting model including the chemical contamination.

6. The method of claim 2, wherein each of the humidity sensor, the particulate matter sensor, the carbon dioxide sensor, and the tVOC sensor are included in the plurality of sensors, and evaluating the medical gas for contamination based on the obtained measurements and the previous measurements obtained over time comprises:
evaluating the medical gas for biological contamination by combining the obtained measurements and the previous measurements obtained over time from the particulate matter sensor, the carbon dioxide sensor, and the tVOC sensor;
evaluating the medical gas for non-biological particulate contamination by combining the obtained measurements and the previous measurements obtained over time from the particulate matter sensor, the carbon dioxide sensor, and the tVOC sensor;
evaluating the medical gas for chemical contamination by combining the obtained measurements and the previous measurements obtained over time from the particulate matter sensor and the tVOC sensor; and
evaluating the medical gas for water vapor contamination based on the obtained measurements and the previous measurements obtained over time from the humidity sensor.

7. The method of claim 2, wherein the obtained measurements and the previous measurements obtained over time comprise aggregate data, and evaluating the medical gas for contamination based on the obtained measurements and the previous measurements obtained over time comprises:
outputting a biological contamination alert responsive to the aggregate data matching a biological contamination model;
outputting a particulate contamination alert responsive to the aggregate data matching a particulate contamination model; and
outputting a chemical contamination alert responsive to the aggregate data matching a chemical contamination model.

8. The method of claim 1, wherein the humidity sensor is included in the plurality of sensors, and the method further comprises outputting a moisture alert responsive to a water vapor content measured by the humidity sensor increasing above a threshold water vapor content.

9. The method of claim 1, wherein outputting the determined gas quality index includes wirelessly transmitting the determined gas quality index to a display of a portable user interface via a remote network.

10. A medical gas quality monitoring system, comprising:
a first gas quality monitor coupled at a first position in a gas flow path, the first gas quality monitor including a plurality of sensors positioned to measure quantities within a medical gas flowing through the gas flow path at the first position;
a user interface including a display; and
a controller including instructions stored in non-transitory memory that, when executed, cause the controller to:
receive measurements from the plurality of sensors of the first gas quality monitor;
determine a gas quality index value using the received measurements;
output the determined gas quality index value to the display; and
output a contamination alert responsive to the gas quality index value being less than a threshold.

11. The medical gas quality monitoring system of claim 10, wherein the first position is internal to a housing of a medical gas flow device positioned at a patient care location.

12. The medical gas quality monitoring system of claim 10, wherein the first position is external to a housing of a medical gas flow device positioned at a patient care location.

13. The medical gas quality monitoring system of claim 10, wherein the first position is at an inlet to a medical gas flow device positioned at a patient care location.

14. The medical gas quality monitoring system of claim 13, further comprising a second gas quality monitor coupled at an outlet of the medical gas flow device, the second gas quality monitor including a second plurality of sensors positioned to measure quantities within the medical gas flowing through the gas flow path at the outlet, and wherein the controller includes further instructions stored in non-transitory memory that, when executed, cause the controller to:
receive measurements from the second plurality of sensors of the second gas quality monitor; and
adjust the gas quality index value using the received measurements from the second gas quality monitor.

15. The medical gas quality monitoring system of claim 10, wherein the first position is at an outlet of a medical gas flow device, upstream of a gas passage configured to couple the outlet to a patient breathing circuit, and external to a housing of the medical gas flow device.

16. The medical gas quality monitoring system of claim 10, wherein the first position is at an outlet of a medical gas flow device, upstream of a gas passage configured to couple the outlet to a patient breathing circuit, and internal to a housing of the medical gas flow device.

17. A system, comprising:
a gas source;
a gas flow device including a patient delivery passage;
a delivery network fluidically coupling the gas source to the gas flow device;
a medical gas quality monitoring system including at least one gas quality monitor, each of the at least one gas quality monitor including each of a plurality of different types of sensors positioned to measure a gas flow originating from the gas source at a location upstream of the patient delivery passage; and
a controller including instructions stored in non-transitory memory that, when executed, cause the controller to:
monitor a quality of the gas flow in real-time based on current measurements received from each of the plurality of different types of sensors; and
evaluate the gas flow for potential contamination in real-time based on the current measurements and previous measurements received from one or more or each of the plurality of different types of sensors.

18. The system of claim 17, wherein the plurality of different types of sensors include a humidity sensor, a volatile organic compound sensor, a particulate matter sensor, and a carbon dioxide sensor.

19. The system of claim 18, wherein the controller further includes a plurality of contamination models stored in non-transitory memory, and to evaluate the gas flow for potential contamination, the controller includes further instructions stored in non-transitory memory that, when executed, cause the controller to:
evaluate the gas flow for potential biological contamination by comparing the current measurements and the previous measurements received from the volatile organic compound sensor, the particulate matter sensor, and the carbon dioxide sensor to a biological contamination model of the plurality of contamination models;
evaluate the gas flow for potential particulate contamination by comparing the current measurements and the previous measurements received from the volatile organic compound sensor, the particulate matter sensor, and the carbon dioxide sensor to a particulate contamination model of the plurality of contamination models; and
evaluate the gas flow for potential chemical contamination by comparing the current measurements and the previous measurements received from the volatile organic compound sensor and the particulate matter sensor to a chemical contamination model of the plurality of contamination models.

20. The system of claim 18, wherein the controller includes further instructions stored in non-transitory memory that, when executed, cause the controller to:
evaluate the gas flow for moisture by comparing the current measurement received from the humidity sensor to a threshold.
